SEQUENCE LISTING (36 sequences)

SEQ ID NO: 1 - protein, 10 aa, synthetic construct
GYTFTSYNMH

SEQ ID NO: 2 - protein, 17 aa, synthetic construct
AIYPGNGDTSYNQKFKG

SEQ ID NO: 3 - protein, 13 aa, synthetic construct
VVYYSNSYWYFDV

SEQ ID NO: 4 - protein, 10 aa, synthetic construct
RASSSVSYMH

SEQ ID NO: 5 - protein, 7 aa, synthetic construct
APSNLAS

SEQ ID NO: 6 - protein, 9 aa, synthetic construct
QQWSFNPPT

SEQ ID NO: 7 - protein, 122 aa, synthetic construct [sequence omitted]

SEQ ID NO: 8 - protein, 106 aa, synthetic construct [sequence omitted]

SEQ ID NO: 9 - protein, 5 aa, synthetic construct
NYYIH

SEQ ID NO: 10 - protein, 17 aa, synthetic construct
WIYPGDGNTKYNEKFKG

SEQ ID NO: 11 - protein, 10 aa, synthetic construct
DSYSNYYFDY

SEQ ID NO: 12 - protein, 17 aa, synthetic construct
KSSQSLLNSRTRKNYLA

SEQ ID NO: 13 - protein, 7 aa, synthetic construct
WASTRES

SEQ ID NO: 14 - protein, 8 aa, synthetic construct
TQSFILRT

SEQ ID NO: 15 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 16 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 17 - protein, 25 aa, synthetic construct [sequence omitted]

SEQ ID NO: 18 - protein, 14 aa, synthetic construct
WVRQAPGKGLEWVG

SEQ ID NO: 19 - protein, 32 aa, synthetic construct
RFTISVDKSKNTLYLQMNSLRAEDTAVYYCAR

SEQ ID NO: 20 - protein, 11 aa, synthetic construct
WGQGTLVTVSS

SEQ ID NO: 21 - protein, 23 aa, synthetic construct
DIQMTQSPSSLSASVGDRVTITC

SEQ ID NO: 22 - protein, 15 aa, synthetic construct
WYQQKPGKAPKPLIY

SEQ ID NO: 23 - protein, 32 aa, synthetic construct
GVPSRFSGSGSGTDFTLTISSLQPEDFATYYC

SEQ ID NO: 24 - protein, 10 aa, synthetic construct
FGQGTKVEIK

SEQ ID NO: 25 - protein, 30 aa, synthetic construct
EVQLVQSGAEVKKPGASVKVSCKASGYTFT

SEQ ID NO: 26 - protein, 14 aa, synthetic construct
WVRQAPGQGLEWIG

SEQ ID NO: 27 - protein, 32 aa, synthetic construct
RATLTADTSTSTAYLELSSLRSEDTAVYYCAR

SEQ ID NO: 28 - protein, 11 aa, synthetic construct
WGQGTLVTVSS

SEQ ID NO: 29 - protein, 23 aa, synthetic construct
DIVMTQSPDSLAVSLGERATINC

SEQ ID NO: 30 - protein, 15 aa, synthetic construct
WYQQKPGQPPKLLIY

SEQ ID NO: 31 - protein, 32 aa, synthetic construct
GVPDRFSGSGSGTDFTLTISSLQAEDVAVYYC

SEQ ID NO: 32 - protein, 10 aa, synthetic construct
FGQGTKVEIK

SEQ ID NO: 33 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 449 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0416391 A1
(43) Pub. Date: Dec. 28, 2023

(54) DOSING FOR TREATMENT WITH ANTI-CD20/ANTI-CD3 BISPECIFIC ANTIBODIES IN ELDERLY PATIENTS

(71) Applicants: Genentech, Inc., South San Francisco, CA (US); Hoffmann-La Roche Inc., Little Falls, NJ (US)

(72) Inventors: Huang HUANG, Mississauga (CA); Carol Elaine O'HEAR, South San Francisco, CA (US); Iris Tranthuyngan TO, South San Francisco, CA (US); Michael Ching-sun WEI, South San Francisco, CA (US); Shen YIN, South San Francisco, CA (US)

(21) Appl. No.: 18/326,605

(22) Filed: May 31, 2023

Related U.S. Application Data
(60) Provisional application No. 63/347,397, filed on May 31, 2022.

(51) Int. Cl. C07K 16/28; A61K 9/00; A61K 31/138; A61K 31/167; A61K 31/573; A61K 39/395; A61P 35/00

(52) U.S. Cl. CPC C07K 16/2887; C07K 16/2809; A61K 9/0019; A61K 31/138; A61K 31/167; A61K 31/573; A61K 39/3955; A61P 35/00; C07K 2317/31; C07K 2317/24; A61K 2039/55

(57) ABSTRACT

The present invention relates to the treatment of elderly subjects (e.g., subjects aged 65 years or older) having relapsed and/or refractory (R/R) non-Hodgkin's lymphoma (NHL). More specifically, the invention pertains to the treatment of subjects having an R/R NHL by intravenous administration of mosunetuzumab.

### SEQUENCE LISTING

The instant application contains a Sequence Listing which has been submitted electronically in XML format and is hereby incorporated by reference in its entirety. Said XML copy, created on May 15, 2023, is named 50474-291002_Sequence_Listing_5_15_23 and is 33,990 bytes in size.

### FIELD OF THE INVENTION

The present invention relates to the treatment of B cell proliferative disorders in elderly subjects. More specifically, the invention pertains to treatment of elderly subjects having a relapsed and/or refractory (R/R) non-Hodgkin's lymphoma (NHL; e.g., an R/R follicular lymphoma (FL), and R/R transformed FL (trFL), or an R/R diffuse large B cell lymphoma (DLBCL)) by administration of a bispecific antibody that binds to anti-cluster of differentiation 20 (CD20) and anti-cluster of differentiation 3 (CD3), e.g., anti-CD20/anti-CD3 bispecific antibody, e.g., mosunetuzumab.

### BACKGROUND

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. Cancer care thus represents a significant and ever-increasing societal burden.

CD20-positive cell proliferative disorders, such as B cell proliferative disorders, are a leading cause of cancer-related deaths. For example, non-Hodgkin's lymphoma (NHL) advances quickly and is fatal if untreated. In the United States, B-cell lymphomas constitute approximately 80%-85% of all cases of NHL. Follicular lymphoma (FL) is the most common indolent subtype of NHL and typically affects elderly patients, with a median age at diagnosis of 65 years and a frequent occurrence in patients aged >75 years (Castellino A, et al. Mediterr J Hematol Infect Dis 2017, 9:e2017009). Aggressive NHLs include DLBCLs, transformed FLs, and Grade 3b FLs. Up to 40% of patients with DLBCL who are treated in the first-line setting will experience disease progression within 3-4 years (Friedberg J W. Hematology Am. Soc. Hematol Educ. Program. 2011, 2011:498-505), and more than half of the patients treated with second-line therapies do not achieve a complete remission (Gisselbrecht C et al. J. Clin. Oncol. 2010, 28:4184-4190).

Bispecific antibodies (e.g., anti-CD20/anti-CD3 bispecific antibodies, e.g., mosunetuzumab) are capable of simultaneously binding cell surface antigens on cytotoxic cells (e.g., T cells, via binding to cluster of differentiation 3 (CD3)) and cancer cells (e.g., B cells, via binding to CD20), wherein the bound cytotoxic cell destroys the juxtaposed bound cancer cell via an immunological synapse. However, use of such antibody-based immunotherapies can be limited by unwanted effects, including cytokine-driven toxicities (e.g., cytokine release syndrome (CRS)), infusion-related reactions (IRRs), severe tumor lysis syndrome (TLS), and central nervous system (CNS) toxicities. Furthermore, since the introduction of monoclonal anti-CD20 antibodies such as rituximab, it has become more challenging to find effective therapies for the large proportion of patients who have prior exposure to anti-CD20 antibodies in their treatments.

Elderly patients (e.g., patients aged about 65 years or older) with FL experience more treatment-related difficulties than younger patients due to increased comorbidities, reduced immune function, and access to fewer treatment options (Castellino A, et al. Mediterr J Hematol Infect Dis 2017; 9:e2017009; Casulo C, et al. Blood Adv 2021; 5:1737-1745). Thus, there is an unmet need in the field for the development of efficacious methods of treating B cell proliferative disorders (e.g., non-Hodgkin's lymphoma (NHL); e.g., follicular lymphoma (FL), transformed FL (trFL), or diffuse large B cell disorder (DLBCL), particularly relapsed and/or refractory (R/R) NHL, FL, trFL, or DLBCL) that achieve a more favorable benefit-risk profile in elderly patients (e.g., patients aged about 65 years or older).

### SUMMARY OF THE INVENTION

The present invention relates to methods of treating a subject (e.g., an elderly subject, e.g., a subject aged about 65 years or older) having a relapsed and/or refractory (R/R) non-Hodgkin's lymphoma (NHL) (e.g., an R/R follicular lymphoma (FL), R/R transformed FL (trFL), or R/R diffuse large B cell lymphoma (DLBCL)) by administration (e.g., intravenous administration) of an anti-CD20/anti-CD3 bispecific antibody (e.g., mosunetuzumab).

The invention is based, in part, on the discovery that dosing regimens involving administration of a bispecific antibody that binds to CD20 and CD3 (e.g., mosunetuzumab) over multiple dosing cycles (e.g., wherein the first dosing cycle is a step-up, fractionated dosing cycle) including a relatively high third dose (C1D3) and/or a dose of a second dosing cycle (C2D1) (“loading doses”) that is greater in amount than a dose of the third dosing cycle (C3D1) and/or additional dosing cycles (“base doses”) can effectively treat elderly subjects (e.g., subjects aged about 65 years or older) having a B cell disorder while reducing toxicity (e.g., cytokine release syndrome and/or severe adverse events). The dosing regimens of the present invention have also been discovered to be particularly efficacious in the treatment of elderly subjects (e.g., subjects aged about 65 years or older).

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having a relapsed or refractory (R/R) non-Hodgkin's lymphoma (NHL; e.g., R/R follicular lymphoma (FL), R/R transformed FL (trFL), or R/R diffuse large B cell lymphoma (DLBCL)) comprising intravenously administering to the subject mosunetuzumab in a dosing regimen comprising at least a first dosing cycle, a second dosing cycle, and a third dosing cycle, wherein: (a) the first three-dose dosing cycle comprises a first dose (C1D1), a second dose (C1D2), and a third dose (C1D3) of mosunetuzumab, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 is about 2 mg (e.g., 2 mg±0.01 mg, 0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third dosing cycle comprises a single dose (C3D1) of mosunetuzumab, wherein the C3D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg). In some embodiments, the C1 D1 is 1±0.2 mg, the C1 D2 is 2±0.4 mg, the C1 D3 is 60±12 mg, the C2D1 is 60±12 mg, and the C3D1 is 30±6 mg.

In some embodiments, the subject has relapsed after, or is refractory to, two or more (e.g., two, three, four, five, six, seven, eight, nine, ten, 11, 12, or more) prior lines of therapy. In some embodiments, the two or more prior lines of therapy comprise an anti-CD20 monoclonal antibody (e.g., rituximab), an alkylating agent (e.g., bendamustine, chlorambucil, cyclophosphamide, ifosfamide, mechlorethamine, melphalan, carmustine, lomustine, streptozocin, busulfan, dacarbazine, temozolomide, altretamine, or thiotepa), or both an anti-CD20 monoclonal antibody and an alkylating agent.

In some embodiments, the R/R NHL is R/R follicular lymphoma (FL), R/R transformed FL (trFL), or R/R diffuse large B cell lymphoma (DLBCL). In a particular embodiment, the R/R NHL is R/R FL. In some embodiments, the R/R FL is histologically documented as Grade 1, 2, or 3a, but not 3b according to the World Health Organization classification of lymphoid neoplasms (as referenced in Swerdlow S H, et al. Blood 2016; 127:2375-90).

In some embodiments, the first, second, and third dosing cycles are 21-day (±1 day) dosing cycles. In some embodiments, the method comprises administering the C1 D1, the C1 D2, and the C1 D3 on Days 1, 8 (±1 day), and 15 (±1 day), respectively, of the first dosing cycle. In some embodiments, the method comprises administering the C2D1 on Day 1 of the second dosing cycle. In some embodiments, the method comprises administering the C3D1 on Day 1 of the third dosing cycle.

In some embodiments, the dosing regimen further comprises one or more (e.g., one, two, three, four, five, six, seven, eight, nine, ten, 11, 12, 13, 14, or more) additional dosing cycles. In some embodiments, the dosing regimen comprises five to 14 (five, six, seven, eight, nine, ten, 11, 12, 13, or 14) additional dosing cycles. In a particular embodiment, the dosing regimen comprises five additional dosing cycles. In another particular embodiment embodiments, the dosing regimen comprises 14 additional dosing cycles. In some embodiments, the one or more (e.g., one, two, three, four, five, six, seven, eight, nine, ten, 11, 12, 13, 14, or more) additional dosing cycles are each 21-day (±1 day) dosing cycles.

In some embodiments, each of the one or more (e.g., one, two, three, four, five, six, seven, eight, nine, ten, 11, 12, 13, 14, or more) of the additional dosing cycles comprises an additional single dose of mosunetuzumab. In some embodiments, the additional single dose of mosunetuzumab is administered to the subject on Day 1 of each respective additional dosing cycle. In some embodiments, the additional single dose of mosunetuzumab is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg). In some embodiments, each additional dose of mosunetuzumab is 30±6 mg.

In some embodiments, mosunetuzumab is administered by intravenous infusion.

In one aspect, the invention provides a method of treating a population of subjects aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to each subject mosunetuzumab in a dosing regimen comprising at least a first 21-day (±1 day) dosing cycle, a second 21-day (±1 day) dosing cycle, and a third 21-day (±1 day) dosing cycle, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third dosing cycle comprises a single dose (C3D1) of mosunetuzumab administered on Day 1 of the third dosing cycle, wherein the C3D1 is about 30 mg (e.g., 30 mg±0.01 mg, ±0.025 mg, ±0.05 mg, 0.075 mg, ±0.1 mg, ±0.2 mg, ±0.3 mg, ±0.4 mg, ±0.5 mg, 0.75 mg, ±1 mg, ±1.5 mg, ±2 mg, or ±3 mg; e.g., 30 mg). In some embodiments, the C1D1 is 1±0.2 mg, the C1D2 is 2±0.4 mg, the C1D3 is 60±12 mg, the C2D1 is 60±12 mg, and the C3D1 is 30±6 mg.

In one aspect, the invention provides a method of treating a population of subjects aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to each subject mosunetuzumab in a dosing regimen comprising eight 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third to eighth dosing cycles each comprises a single dose (C3D1-C8D1) of mosunetuzumab administered on Day 1 of each respective dosing cycle, wherein each of the C3D1-C8D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg). In some embodiments, the C1 D1 is 1±0.2 mg, the C1 D2 is 2±0.4 mg, the C1 D3 is 60±12 mg, the C2D1 is 60±12 mg, and each of the C3D1-C8D1 is 30±6 mg.

In one aspect, the invention provides a method of treating a population of subjects aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to each subject mosunetuzumab in a dosing regimen comprising 17 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third to 17th dosing cycles each comprises a single dose (C3D1-C17D1) of mosunetuzumab administered on Day 1 of each respective dosing cycle, wherein each of the C3D1-C17D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg). In some embodiments, the C1 D1 is 1±0.2 mg, the C1 D2 is 2±0.4 mg, the C1 D3 is 60±12 mg, the C2D1 is 60±12 mg, and each of the C3D1-C17D1 is 30±6 mg.

In some embodiments, the objective response rate in the population of subjects is 65% (e.g., ≥70%, 75%, 80%, 85%, 90%, or 95%; e.g., 65-100%, 70-100%, 75-100%, 80-100%, 85-100%, 90-100%, 95-100%, 65-90%, 65-80%, 65-70%, 75-90%, 75-85%, 70-90%, or 80-90%; e.g., about 65%, about 70%, about 75%, about 80%, about 85%, about 90%, about 95%, or about 100%). In some embodiments, the objective response rate in the population of subjects is 75%. In some embodiments, the objective response rate in the population of subjects is 85%. In some embodiments, the objective response rate in the population of subjects is 90%.

In some embodiments, the complete response rate in the population of subjects is 50% (e.g., ≥55%, 60%, 65%, 70%, 75%, 80%, 85%, 90%, or 95%; e.g., 50-100%, 60-100%, 70-100%, 80-100%, 90-100%, 95-100%, 50-90%, 50-80%, 50-70%, 50-60%, 50%-75%, 75-90%, 60-80%, 75-85%, 70-90%, or 80-90%; e.g., about 50%, about 55%, about 60%, about 65%, about 70%, about 75%, about 80%, about 85%, about 90%, about 95%, or about 100%). In some embodiments, the complete response rate in the population of subjects is 60%. In some embodiments, the complete response rate in the population of subjects is 70%.

In some embodiments, 40% (e.g., 45%, 50%, 55%, 60%, 65%, 70%, 75%, 80%, 85%, 90%, or 95%; e.g., 40-100%, 50-100%, 60-100%, 70-100%, 80-100%, 90-100%, 95-100%, 40-90%, 40-80%, 40-70%, 40-60%, 40-50%, 50%-70%, 50-60%, 75-90%, 60-80%, 75-85%, 70-90%, or 80-90%; e.g., about 40%, about 45%, about 50%, about 55%, about 60%, about 65%, about 70%, about 75%, about 80%, about 85%, about 90%, about 95%, or about 100%) of subjects having an objective response maintained remission for 18 months. In some embodiments, 50% of subjects having an objective response maintained remission for 18 months.

In some embodiments, 50% (e.g., 55%, 60%, 65%, 70%, 75%, 80%, 85%, 90%, or 95%; e.g., 50-100%, 60-100%, 70-100%, 80-100%, 90-100%, 95-100%, 50-90%, 50-80%, 50-70%, 50-60%, 50%-75%, 75-90%, 60-80%, 75-85%, 70-90%, or 80-90%; e.g., about 50%, about 55%, about 60%, about 65%, about 70%, about 75%, about 80%, about 85%, about 90%, about 95%, or about 100%) of subjects having a complete response maintained complete remission for 18 months. In some embodiments, 60% of subjects having a complete response maintained complete remission for 18 months.

In some embodiments, the median duration of response in the population of subjects is 9 months (e.g., 9, 10, 11, 12, 13, 14, 15, 18, 21, 24, 27, 30, 33, 36 months, or more; e.g., 9-12 months, 9-15 months, 9-18 months, 9-24 months, 12-18 months, 12-24 months, 12-36 months, 18-36 months, 24-36 months, or more; e.g., 10, 11, 12, 13, 14, 15, 16, 17, 18, 19, 20, 21, 24, 27, 30, 33, 36 months, or more). In some embodiments, the median duration of response in the population of subjects is 15 months. In some embodiments, the median duration of response in the population of subjects is 18 months.

In some embodiments, the median duration of complete response in the population of subjects is 12 months (e.g., 12, 13, 14, 15, 16, 17, 18, 19, 20, 21, 24, 27, 30, 33, 36 months, or more; e.g., 12-15 months, 12-18 months, 12-21 months, 12-24 months, 12-30 months, 12-36 months, 15-18 months, 15-24 months, 18-24 months, 24-36 months, 18-36 months, or more; e.g., 12, 13, 14, 15, 16, 17, 18, 19, 20, 21, 24, 27, 30, 33, 36 months, or more). In some embodiments, the median duration of complete response in the population of subjects is 16 months. In some embodiments, the median duration of complete response in the population of subjects is 18 months.

In some embodiments, 35% of subjects (e.g., 40%, 45%, 50%, 55%, 60%, 65%, 70%, 75%, 80%, 85%, 90%, or 95%; e.g., 35-100%, 40-100%, 50-100%, 60-100%, 70-100%, 80-100%, 90-100%, 95-100%, 35-90%, 35-80%, 35-70%, 35-60%, 35-50%, 35-45%, 40-80%, 40-60%, 50%-70%, 50-60%, 75-90%, 60-80%, 75-85%, 70-90%, or 80-90%; e.g., about 35%, about 40%, about 45%, about 50%, about 55%, about 60%, about 65%, about 70%, about 75%, about 80%, about 85%, about 90%, about 95%, or about 100%) have a progression-free survival of 18 months. In some embodiments, 45% of subjects have a progression-free survival of 18 months.

In some embodiments, the median progression-free survival in the population of subjects is 12 months (e.g., 12, 13, 14, 15, 16, 17, 18, 19, 20, 21, 24, 27, 30, 33, 36 months, or more; e.g., 12-15 months, 12-18 months, 12-21 months, 12-24 months, 12-30 months, 12-36 months, 15-18 months, 15-24 months, 18-24 months, 24-36 months, 18-36 months, or more; e.g., 12, 13, 14, 15, 16, 17, 18, 19, 20, 21, 24, 27, 30, 33, 36 months, or more). In some embodiments, the median progression-free survival in the population of subjects is 17 months.

In some embodiments, the rate of serious adverse events, excluding Grade 5 malignant neoplasm progression, in the population of subjects is ≤45% (e.g., ≤40%, 35%, 30%, 25%, 20%, 15%, 10%, or 5%; e.g., 0-45%, 0-35%, 0-25%, 0-15%, 0-10%, 0-5%, 5-15%, 15-30%, 30-45%, 10-30%, 20-40%, 35-45%, 30-40%, 20-30%, or 10-20%; e.g., about 0%, about 5%, about 10%, about 15%, about 20%, about 25%, about 30%, about 35%, about 40% or about 45%). In some embodiments, the rate of serious adverse events, excluding Grade 5 malignant neoplasm progression, in the population of subjects is ≤40%. In some embodiments, the rate of serious adverse events, excluding Grade 5 malignant neoplasm progression, in the population of subjects is ≤50% (e.g., about 50%, about 49%, about 48%, about 47%, or about 46%).

In some embodiments, the rate of serious adverse events related to mosunetuzumab in the population of subjects is ≤40% (e.g., ≤35%, 30%, 25%, 20%, 15%, 10%, or 5%; e.g., 0-40%, 0-35%, 0-25%, 0-15%, 0-10%, 0-5%, 5-15%, 15-30%, 30-40%, 10-30%, 20-40%, 30-40%, 20-30%, or 10-20%; e.g., about 0%, about 5%, about 10%, about 15%, about 20%, about 25%, about 30%, about 35%, or about 40%). In some embodiments, the rate of serious adverse events related to mosunetuzumab in the population of subjects is ≤30%. In some embodiments, the rate of serious adverse events related to mosunetuzumab in the population of subjects is ≤35%.

In some embodiments, the rate of cytokine release syndrome having a grade of 3 or higher (as defined by the American Society for Transplantation and Cellular Therapy, 2018; ASTCT) in the population of subjects is ≤5% (e.g., ≤4%, 3%, 2%, or 1%; e.g., 0-5%, 1-5%, 2-5%, 3-5%, 4-5%, 1-3%, 2-4%, or 0-3%; e.g., about 0%, about 1%, about 2%, about 3%, about 4%, or about 5%). In some embodiments, the rate of cytokine release syndrome having a grade of 3 or higher (as defined by the ASTCT) in the population of subjects is ≤3%.

In some embodiments, the rate of cytokine release syndrome of any grade (as defined by the ASTCT) in the population of subjects is ≤40% (e.g., ≤35%, 30%, 25%, 20%, 15%, 10%, or 5%; e.g., 0-40%, 0-35%, 0-25%, 0-15%, 0-10%, 0-5%, 5-15%, 15-30%, 25-40%, 10-30%, 20-40%, 25-35%, 35-40%, 30-40%, 20-30%, or 10-20%; e.g., about 0%, about 5%, about 10%, about 15%, about 20%, about 25%, about 30%, about 35%, or about 40%). In some embodiments, the rate of cytokine release syndrome of any grade (as defined by the ASTCT) in the population of subjects is ≤30%. In some embodiments, the rate of cytokine release syndrome of any grade (as defined by the ASTCT) in the population of subjects is ≤50% (e.g., ≤45%; e.g., 40-50%, 30-50%, 25-50%, 20-50%, 10-50%, 0-50%, 40-45%, 30-35%, 20-45%, 10-45%, 0-45%; e.g., about 41%, about 42%, about 43%, about 44%, about 45%, about 46%, about 47%, about 48%, about 49%, or about 50%).

In one aspect, the invention provides a method of achieving an objective response, a complete response, or progression-free survival in a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL), wherein the method comprises intravenously administering to the subject mosunetuzumab in a dosing regimen comprising at least a first 21-day (±1 day) dosing cycle, a second 21-day (±1 day) dosing cycle, and a third 21-day (±1 day) dosing cycle, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, 0.075 mg, ±0.1 mg, ±0.2 mg, ±0.3 mg, +0.4 mg, ±0.5 mg, 0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third dosing cycle comprises a single dose (C3D1) of mosunetuzumab administered on Day 1 of the third dosing cycle, wherein the C3D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg). In some embodiments, the C1 D1 is 1±0.2 mg, the C1 D2 is 2±0.4 mg, the C1 D3 is 60±12 mg, the C2D1 is 60±12 mg, and the C3D1 is 30±6 mg.

In one aspect, the invention provides a method of achieving an objective response, a complete response, or progression-free survival in a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL), wherein the method comprises intravenously administering to the subject mosunetuzumab in a dosing regimen comprising eight 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, 0.075 mg, or ±0.1 mg; e.g., 1 mg), the C1 D2 about 2 mg (e.g., 2 mg±0.01 mg, ±0.025 mg, ±0.05 mg, 0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, +2 mg, ±3 mg, ±4 mg, ±5 mg, ±6 mg; e.g., 60 mg); and (c) the third to eighth dosing cycles each comprises a single dose (C3D1-C8D1) of mosunetuzumab administered on Day 1 of each respective dosing cycle, wherein each of the C3D1-C8D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg). In some embodiments, the C1 D1 is 1±0.2 mg, the C1 D2 is 2±0.4 mg, the C1 D3 is 60±12 mg, the C2D1 is 60±12 mg, and each of the C3D1-C8D1 is 30±6 mg.

In one aspect, the invention provides a method of achieving an objective response, a complete response, or progression-free survival in a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL), wherein the method comprises intravenously administering to the subject mosunetuzumab in a dosing regimen comprising 17 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, 0.075 mg, or ±0.1 mg; e.g., 1 mg), the C1 D2 about 2 mg (e.g., 2 mg±0.01 mg, ±0.025 mg, ±0.05 mg, 0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, +2 mg, ±3 mg, ±4 mg, ±5 mg, ±6 mg; e.g., 60 mg); and (c) the third to 17th dosing cycles each comprises a single dose (C3D1-C17D1) of mosunetuzumab administered on Day 1 of each respective dosing cycle, wherein each of the C3D1-C17D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, 0.75 mg, ±1 mg, ±1.5 mg, ±2 mg, or ±3 mg; e.g., 30 mg). In some embodiments, the C1 D1 is 1±0.2 mg, the C1 D2 is 2±0.4 mg, the C1 D3 is 60±12 mg, the C2D1 is 60±12 mg, and each of the C3D1-C17D1 is 30±6 mg.

In some embodiments, the subject has relapsed after, or is refractory to, two or more (e.g., two, three, four, five, six, seven, eight, nine, ten, 11, 12, or more) prior lines of therapy. In some embodiments, the two or more prior lines of therapy comprise an anti-CD20 monoclonal antibody (e.g., rituximab), an alkylating agent (e.g., bendamustine, chlorambucil, cyclophosphamide, ifosfamide, mechlorethamine, melphalan, carmustine, lomustine, streptozocin, busulfan, dacarbazine, temozolomide, altretamine, or thiotepa), or both an anti-CD20 monoclonal antibody and an alkylating agent.

In some embodiments, the R/R NHL is R/R follicular lymphoma (FL), R/R transformed FL (trFL), or R/R diffuse large B cell lymphoma (DLBCL). In some embodiments, the R/R NHL is R/R FL. In some embodiments, the R/R FL is histologically documented as Grade 1, 2, or 3a, but not 3b according to the World Health Organization classification of lymphoid neoplasms (as referenced in Swerdlow S H, et al. Blood 2016; 127:2375-90).

In some embodiments, mosunetuzumab is administered by intravenous infusion.

In some embodiments, the subject achieves an objective response. In some embodiments, the subject achieves a complete response. In some embodiments, the progression-free survival is maintained for ≥12 months (e.g., ≥12, 13, 14, 15, 18, 21, 24, 27, 30, 33, 36 months, or more; e.g., 12-15 months, 12-18 months, 12-21 months, 12-24 months, 12-30 months, 12-36 months, 15-18 months, 15-24 months, 18-24 months, 24-36 months, 18-36 months, or more; e.g., 12, 13, 14, 15, 16, 17, 18, 19, 20, 21, 24, 27, 30, 33, 36 months, or more). In some embodiments, the progression-free survival is maintained for ≥18 months.

In some embodiments, the method further comprises administering to the subject or to each subject in the population of subjects one or more (e.g., one, two, three, four, five, six, or more) additional therapeutic agents.

In some embodiments, the one or more (e.g., one, two, three, four, five, six, or more) additional therapeutic agents comprises tocilizumab.

In some embodiments, the one or more (e.g., one, two, three, four, five, six, or more) additional therapeutic agents comprises a corticosteroid. In some embodiments, the corticosteroid comprises dexamethasone or methylprednisolone. In some embodiments, the corticosteroid is administered to the subject or to each subject in the population of subjects at least one hour (e.g., 60 70, 80, 90, 120, 150, 180, 210, 240, 300 minutes, or more) prior to the administration of any dose of mosunetuzumab. In some embodiments, the corticosteroid is only administered during dosing cycle 1 or dosing cycle 2. In some embodiments, the corticosteroid is additionally administered during one or more dosing cycles after dosing cycle 2 (e.g., in dosing cycle 3, 4, 5, 6, 7, 8, 9, 10, 11, 12, 13, 14, 15, 16, and/or 17). In some embodiments, the corticosteroid is administered intravenously. In some embodiments, the corticosteroid comprises dexamethasone and is administered at a dose of about 20 mg (e.g., 20 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, or ±2 mg; e.g., 20 mg). In some embodiments, the corticosteroid comprises methylprednisolone and is administered at a dose of about 80 mg (e.g., 80 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±8 mg, ±7 mg, or ±8 mg; e.g., 80 mg).

In some embodiments, the one or more (e.g., one, two, three, four, five, six, or more) additional therapeutic agents comprises an anti-histamine. In some embodiments, the anti-histamine is administered to the subject or to each subject in the population of subjects at least 30 minutes (e.g., 30, 35, 40, 45, 50, 60 70, 80, 90, 120, 150, 180 minutes, or more) prior to the administration of any dose of mosunetuzumab. In some embodiments, the anti-histamine is only administered during dosing cycle 1 or dosing cycle 2. In some embodiments, the anti-histamine is additionally administered during one or more dosing cycles after dosing cycle 2 (e.g., in dosing cycle 3, 4, 5, 6, 7, 8, 9, 10, 11, 12, 13, 14, 15, 16, and/or 17). In some embodiments, the anti-histamine is administered orally or intravenously. In some embodiments, the anti-histamine comprises diphenhydramine hydrochloride and is administered at a dose of about 50-100 mg (e.g., 50-90 mg, 50-80 mg, 50-70 mg, 50-60 mg, 60-100 mg, 70-100 mg, 80-100 mg, 90-100 mg, 70-80 mg, 60-90 mg, 60-80 mg, 70-90 mg, or 65-85 mg; e.g., about 50 mg, about 55 mg, about 60 mg, about 65 mg, about 70 mg, about 75 mg, about 80 mg, about 85 mg, about 90 mg, about 95 mg, or about 100 mg).

In some embodiments, the one or more (e.g., one, two, three, four, five, six, or more) additional therapeutic agents comprises an anti-pyretic. In some embodiments, the anti-pyretic is administered to the subject or to each subject in the population of subjects at least 30 minutes (e.g., 30, 35, 40, 45, 50, 60 70, 80, 90, 120, 150, 180 minutes, or more) prior to the administration of any dose of mosunetuzumab. In some embodiments, the anti-pyretic is only administered during dosing cycle 1 or dosing cycle 2. In some embodiments, the anti-pyretic is additionally administered during one or more dosing cycles after dosing cycle 2 (e.g., in dosing cycle 3, 4, 5, 6, 7, 8, 9, 10, 11, 12, 13, 14, 15, 16, and/or 17). In some embodiments, the anti-pyretic is administered orally. In some embodiments, the anti-pyretic comprises acetaminophen and is administered at a dose of about 500-1000 mg (e.g., 500-900 mg, 500-800 mg, 500-700 mg, 500-600 mg, 600-1000 mg, 700-1000 mg, 800-1000 mg, 900-1000 mg, 700-800 mg, 600-900 mg, 600-800 mg, 700-900 mg, or 650-850 mg; e.g., about 500 mg, about 550 mg, about 600 mg, about 650 mg, about 700 mg, about 750 mg, about 800 mg, about 850 mg, about 900 mg, about 950 mg, or about 1000 mg).

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to the subject mosunetuzumab in a dosing regimen comprising at least a first 21-day (±1 day) dosing cycle, a second 21-day (±1 day) dosing cycle, and a third 21-day (±1 day) dosing cycle, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, 0.075 mg, ±0.1 mg, ±0.2 mg, ±0.3 mg, +0.4 mg, ±0.5 mg, 0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third dosing cycle comprises a single dose (C3D1) of mosunetuzumab administered on Day 1 of the third dosing cycle, wherein the C3D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy. In some embodiments, the C1 D1 is 1±0.2 mg, the C1 D2 is 2±0.4 mg, the C1 D3 is 60±12 mg, the C2D1 is 60±12 mg, and the C3D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to the subject mosunetuzumab in a dosing regimen comprising eight 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, 0.075 mg, or ±0.1 mg; e.g., 1 mg), the C1 D2 about 2 mg (e.g., 2 mg±0.01 mg, ±0.025 mg, ±0.05 mg, 0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, +2 mg, ±3 mg, ±4 mg, ±5 mg, ±6 mg; e.g., 60 mg); and (c) the third to eighth dosing cycles each comprises a single dose (C3D1-C8D1) of mosunetuzumab administered on Day 1 of each respective dosing cycle, wherein each of the C3D1-C8D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy. In some embodiments, the C1 D1 is 1±0.2 mg, the C1 D2 is 2±0.4 mg, the C1 D3 is 60±12 mg, the C2D1 is 60±12 mg, and each of the C3D1-C8D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to the subject mosunetuzumab in a dosing regimen comprising 17 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, 0.075 mg, or ±0.1 mg; e.g., 1 mg), the C1 D2 about 2 mg (e.g., 2 mg±0.01 mg, ±0.025 mg, ±0.05 mg, 0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, +2 mg, ±3 mg, ±4 mg, ±5 mg, ±6 mg; e.g., 60 mg); and (c) the third to 17th dosing cycles each comprises a single dose (C3D1-C17D1) of mosunetuzumab administered on Day 1 of each respective dosing cycle, wherein each of the C3D1-C17D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, 0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy. In some embodiments, the C1 D1 is 1±0.2 mg, the C1 D2 is 2±0.4 mg, the C1 D3 is 60±12 mg, the C2D1 is 60±12 mg, and each of the C3D1-C17D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to the subject mosunetuzumab in a dosing regimen comprising at least a first 21-day (±1 day) dosing cycle, a second 21-day (±1 day) dosing cycle, and a third 21-day (±1 day) dosing cycle, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, 0.075 mg, ±0.1 mg, ±0.2 mg, ±0.3 mg, +0.4 mg, ±0.5 mg, 0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third dosing cycle comprises a single dose (C3D1) of mosunetuzumab administered on Day 1 of the third dosing cycle, wherein the C3D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy, and wherein the dosing regimen further comprises: (i) administering a corticosteroid, wherein a single dose of the corticosteroid is administered to the subject at least one hour (e.g., 60 70, 80, 90, 120, 150, 180, 210, 240, 300 minutes, or more) prior to the administration of each of the C1 D1, C1 D2, C1 D3, and C2D1 of mosunetuzumab; (ii) administering an anti-histamine, wherein a single dose of the anti-histamine is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab; and/or (iii) administering an anti-pyretic, wherein a single dose of the anti-pyretic is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab. In some embodiments, the C1 D1 is 1±0.2 mg, the C1 D2 is 2±0.4 mg, the C1 D3 is 60±12 mg, the C2D1 is 60±12 mg, and the C3D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to the subject mosunetuzumab in a dosing regimen comprising eight 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, 0.075 mg, or ±0.1 mg; e.g., 1 mg), the C1 D2 about 2 mg (e.g., 2 mg±0.01 mg, ±0.025 mg, ±0.05 mg, 0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, +2 mg, ±3 mg, ±4 mg, ±5 mg, ±6 mg; e.g., 60 mg); and (c) the third to eighth dosing cycles each comprises a single dose (C3D1-C8D1) of mosunetuzumab administered on Day 1 of each respective dosing cycle, wherein each of the C3D1-C8D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more prior (e.g., two, three, four, five, six, seven, eight, nine, ten, 11, 12, or more) lines of therapy, and wherein the dosing regimen further comprises: (i) administering a corticosteroid, wherein a single dose of the corticosteroid is administered to the subject at least one hour (e.g., 60 70, 80, 90, 120, 150, 180, 210, 240, 300 minutes, or more) prior to the administration of each of the C1 D1, C1 D2, C1 D3, and C2D1 of mosunetuzumab; (ii) administering an anti-histamine, wherein a single dose of the anti-histamine is administered to the subject at least 30 (e.g., 30, 35, 40, 45, 50, 60 70, 80, 90, 120, 150, 180 minutes, or more) minutes prior to the administration of each of the C1 D1, C1 D2, C1 D3, and C2D1 of mosunetuzumab; and/or (iii) administering an anti-pyretic, wherein a single dose of the anti-pyretic is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab. In some embodiments, the C1 D1 is 1±0.2 mg, the C1D2 is 2±0.4 mg, the C1D3 is 60±12 mg, the C2D1 is 60±12 mg, and each of the C3D1-C8D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to the subject mosunetuzumab in a dosing regimen comprising 17 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, 0.075 mg, or ±0.1 mg; e.g., 1 mg), the C1 D2 about 2 mg (e.g., 2 mg±0.01 mg, ±0.025 mg, ±0.05 mg, 0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, +2 mg, ±3 mg, ±4 mg, ±5 mg, ±6 mg; e.g., 60 mg); and (c) the third to 17th dosing cycles each comprises a single dose (C3D1-C17D1) of mosunetuzumab administered on Day 1 of each respective dosing cycle, wherein each of the C3D1-C17D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, 0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy, and wherein the dosing regimen further comprises: (i) administering a corticosteroid, wherein a single dose of the corticosteroid is administered to the subject at least one hour (e.g., 60 70, 80, 90, 120, 150, 180, 210, 240, 300 minutes, or more) prior to the administration of each of the C1 D1, C1 D2, C1 D3, and C2D1 of mosunetuzumab; (ii) administering an anti-histamine, wherein a single dose of the anti-histamine is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab; and/or (iii) administering an anti-pyretic, wherein a single dose of the anti-pyretic is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab. In some embodiments, the C1 D1 is 1±0.2 mg, the C1 D2 is 2±0.4 mg, the C1 D3 is 60±12 mg, the C2D1 is 60±12 mg, and each of the C3D1-C17D1 is 30±6 mg.

In some embodiments, (i) a single dose of the corticosteroid is administered prior to the administration of any dose of mosunetuzumab in any of the one or more of any of the dosing cycles after the second dosing cycle (e.g., prior to administration of the C3D1, C4D1, C5D1, C6D1, C7D1, C8D1, C9D1, C10D1, C11 D1, C12D1, C13D1, and/or C14D1 of mosunetuzumab); (ii) a single dose of the anti-histamine is administered prior to the administration of any dose of mosunetuzumab in any of the one or more of any of the dosing cycles after the second dosing cycle (e.g., prior to administration of the C3D1, C4D1, C5D1, C6D1, C7D1, C8D1, C9D1, C10D1, C11D1, C12D1, C13D1, and/or C14D1 of mosunetuzumab); and/or (iii) a single dose of the anti-pyretic is administered prior to the administration of any dose of mosunetuzumab in any of the one or more of any of the dosing cycles after the second dosing cycle (e.g., prior to administration of the C3D1, C4D1, C5D1, C6D1, C7D1, C8D1, C9D1, C10D1, C11 D1, C12D1, C13D1, and/or C14D1 of mosunetuzumab).

In some embodiments, (i) the corticosteroid is administered intravenously; (ii) the anti-histamine is administered orally or intravenously; and/or (iii) the anti-pyretic is administered orally.

In some embodiments, (i) the corticosteroid comprises dexamethasone and is administered at a dose of about 20 mg (e.g., 20 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, or ±2 mg; e.g., 20 mg) or the corticosteroid comprises methylprednisolone and is administered at a dose of about 80 mg (e.g., 80 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±8 mg, ±7 mg, or ±8 mg; e.g., 80 mg); (ii) the anti-histamine comprises diphenhydramine hydrochloride and is administered at a dose of about 50-100 mg (e.g., 50-90 mg, 50-80 mg, 50-70 mg, 50-60 mg, 60-100 mg, 70-100 mg, 80-100 mg, 90-100 mg, 70-80 mg, 60-90 mg, 60-80 mg, 70-90 mg, or 65-85 mg; e.g., about 50 mg, about 55 mg, about 60 mg, about 65 mg, about 70 mg, about 75 mg, about 80 mg, about 85 mg, about 90 mg, about 95 mg, or about 100 mg); and/or (iii) the anti-pyretic comprises acetaminophen and is administered at a dose of about 500-1000 mg (e.g., 500-900 mg, 500-800 mg, 500-700 mg, 500-600 mg, 600-1000 mg, 700-1000 mg, 800-1000 mg, 900-1000 mg, 700-800 mg, 600-900 mg, 600-800 mg, 700-900 mg, or 650-850 mg; e.g., about 500 mg, about 550 mg, about 600 mg, about 650 mg, about 700 mg, about 750 mg, about 800 mg, about 850 mg, about 900 mg, about 950 mg, or about 1000 mg).

In some embodiments, the subject described herein is a human subject.

### BRIEF DESCRIPTION OF THE DRAWING

FIG. 1 is a schematic diagram showing the overview of a step-load-base dosing of mosunetuzumab. The vertical bars indicate the relative amount of administered mosunetuzumab. Described is a step load-base dosing of 1/2/60/60/30 mg. Patients are administered a first dose (C1D1 dose; i.e., a first step dose) of about 1 mg mosunetuzumab on Day 1 of dosing cycle (Cycle) 1, followed by a second dose (C1D2 dose; i.e., a second step dose) of about 2 mg mosunetuzumab on Day 8 of Cycle 1. The patients are then administered a third dose (C1D3 dose; i.e., a first loading dose) of about 60 mg mosunetuzumab on Day 15 of Cycle 1, followed by first dose (C2D1 dose; i.e., a second loading dose) of about 60 mg mosunetuzumab on Day 1 of Cycle 2. Thereafter, the patient is administered base doses of about 30 mg mosunetuzumab on Day 1 of each subsequent dosing cycle (e.g., C3D1 dose, C4D1 dose, etc.). The patients are initially administered 6 doses (i.e., base doses) on Day 1 of Cycle 3 (C3D1) to Day 1 of Cycle 8 (C8D1). Patients who do not achieve CR following 8 dosing cycles of treatment continue to receive base doses of about 30 mg mosunetuzumab for 8 or 17 additional cycles of treatment. ECOG PS, Eastern Cooperative Oncology Group performance status; FL, follicular lymphoma; R/R, relapsed/refractory.

### DETAILED DESCRIPTION

The present invention involves methods of treating an elderly subject (i.e., aged about 65 years or older) or a population of elderly subjects (i.e., each aged about 65 years or older) having a relapsed and/or refractory (R/R) non-Hodgkin's lymphoma (NHL; e.g., a diffuse large B cell lymphoma (DLBCL; e.g., a Richter's Transformation), a follicular lymphoma (FL; e.g., a Grade 1 FL, a Grade 2 FL, a Grade 3 FL (e.g., a Grade 3a FL, or a Grade 3b FL), or a transformed FL (trFL); e.g., an R/R NHL, R/R DLBCL, R/R FL, or R/R trFL) by intravenously administering (e.g., via intravenous infusion) to the subject an anti-CD20/anti-CD3 bispecific antibody (e.g., mosunetuzumab) in a dosing regimen comprising at least a first dosing cycle, a second dosing cycle, and a third dosing cycle. In some instances, the first dosing cycle comprises a first dose (C1D1), a second dose (C1D2), and a third dose (C1D3) of mosunetuzumab, wherein the C1 D1 is from about 1 mg, the C1 D2 is about 2 mg, and the C1 D3 is about 60 mg. In some instances, the second dosing cycle includes a single dose (C2D1) of mosunetuzumab of about 60 mg. In some instances, the third dosing cycle includes a single dose (C3D1) of mosunetuzumab of about 30 mg.

As previously noted, the invention is based, in part, on the discovery that dosing regimens involving administration of mosunetuzumab over multiple dosing cycles (e.g., wherein the first dosing cycle is a step-up, fractionated dosing cycle) including a relatively high third dose (C1D3) and/or a dose of a second dosing cycle (C2D1) that is greater in amount than a dose of the third dosing cycle (C3D1) and/or additional dosing cycles can effectively treat subjects having an R/R NHL (e.g., R/R FL, R/R DLBCL, or R/R trFL) while reducing toxicity (e.g., serious adverse events and/or cytokine release syndrome). In addition, the invention is based, in part, one the discovery that the dosing regimens disclosed herein are particularly effective for elderly subjects (i.e., subjects aged about 65 years or older), e.g., when compared to non-elderly subjects (i.e., subjects aged about 64 years or younger, e.g., subject aged about 18-64).

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al., eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### II. Definitions

It is to be understood that aspects and embodiments of the invention described herein include “comprising,” “consisting,” and “consisting essentially of” aspects and embodiments.

As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. In some embodiments, the term “about” a value or parameter refers to that value or parameter ±10%. In a particular embodiment, when referring to the age of a subject or individual, the term “about” refers to the range from the age to the age +1 (i.e., a subject who is 65 years old is “about 65 years old” until the subject is 66 years old).

A “disorder” is any condition that would benefit from treatment including, but not limited to, chronic and acute disorders or diseases including those pathological conditions which predispose the mammal to the disorder in question.

The terms “cell proliferative disorder” and “proliferative disorder” refer to disorders that are associated with some degree of abnormal cell proliferation. In one embodiment, the cell proliferative disorder is cancer. In another embodiment, the cell proliferative disorder is a tumor.

The terms “cancer” and “cancerous” refer to or describe the physiological condition in mammals that is typically characterized by unregulated cell growth. Examples of cancer include, but are not limited to, hematologic cancers, such as mature B cell cancers, excluding Hodgkin's lymphoma, but including non-Hodgkin's lymphoma (NHL), such as diffuse large B cell lymphoma (DLBCL), which may be relapsed and/or refractory DLBCL or a Richter's transformation. Other specific examples of cancer also include germinal-center B cell-like (GCB) diffuse large B cell lymphoma (DLBCL), activated B cell-like (ABC) DLBCL, follicular lymphoma (FL), transformed FL, mantle cell lymphoma (MCL), acute myeloid leukemia (AML), chronic lymphoid leukemia (CLL), marginal zone lymphoma (MZL), transformed MZL, high grade B-cell lymphoma, primary mediastinal (thymic) large B cell lymphoma (PMLBCL), small lymphocytic leukemia (SLL), lymphoplasmacytic lymphoma (LL), transformed LL, Waldenstrom macroglobulinemia (WM), central nervous system lymphoma (CNSL), Burkitt's lymphoma (BL), B cell prolymphocytic leukemia, splenic marginal zone lymphoma, hairy cell leukemia, splenic lymphoma/leukemia, unclassifiable, splenic diffuse red pulp small B cell lymphoma, hairy cell leukemia variant, heavy chain diseases, a heavy chain disease, γ heavy chain disease, μ heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, primary cutaneous follicle center lymphoma, T cell/histiocyte rich large B cell lymphoma, primary DLBCL of the CNS, primary cutaneous DLBCL, leg type, EBV-positive DLBCL of the elderly, DLBCL associated with chronic inflammation, lymphomatoid granulomatosis, intravascular large B cell lymphoma, ALK-positive large B cell lymphoma, plasmablastic lymphoma, large B cell lymphoma arising in HHV8-associated multicentric Castleman disease, primary effusion lymphoma: B cell lymphoma, unclassifiable, with features intermediate between DLBCL and Burkitt lymphoma, and B cell lymphoma, unclassifiable, with features intermediate between DLBCL and classical Hodgkin's lymphoma. Further examples of cancer include, but are not limited to, carcinoma, lymphoma, blastoma, sarcoma, and leukemia or lymphoid malignancies, including B cell lymphomas. More particular examples of such cancers include, but are not limited to, multiple myeloma (MM); low-grade/follicular NHL; small lymphocytic (SL) NHL; intermediate-grade/follicular NHL; intermediate-grade diffuse NHL; high-grade immunoblastic NHL; high-grade lymphoblastic NHL; high-grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD). In a particular embodiment, the cancer is an NHL. In some embodiments, the NHL is relapsed and/or refractory (R/R). In some embodiments, the R/R NHL is an R/R FL, an R/R DLBCL, or an R/R transformed FL (trFL). In a particular embodiment, the R/R NHL is an R/R FL (e.g., a Grade 1, 2, or 3a, but not 3b, R/R FL according to the World Health Organization classification of lymphoid neoplasms (as referenced in Swerdlow S H, et al. Blood 2016; 127:2375-90)).

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “B cell proliferative disorder” or “B cell malignancy” refer to disorders that are associated with some degree of abnormal B cell proliferation and include, for example, lymphomas, leukemias, myelomas, and myelodysplastic syndromes. In some instances, the B cell proliferative disorder is a lymphoma, such as non-Hodgkin's lymphoma (NHL), including, for example, follicular lymphoma (FL) (e.g., a relapsed and/or refractory FL or transformed FL (trFL)), diffuse large B cell lymphoma (DLBCL) (e.g., a relapsed and/or refractory DLBCL or a Richter's transformation), mantle cell lymphoma (MCL), high grade B-cell lymphoma (HGBL), primary mediastinal (thymic) large B-cell lymphoma (PMLBCL), diffuse B cell lymphoma, small lymphocytic lymphoma, marginal zone lymphoma (MZL), Burkitt lymphoma, or lymphoplasmacytic lymphoma. In another embodiment, the B cell proliferative disorder is a leukemia, such as chronic lymphocytic leukemia (CLL). In some embodiments, the B-cell proliferative disorder (e.g., NHL) is relapsed and/or refractory (R/R). In some embodiments, the R/R B-cell proliferative disorder (e.g., NHL), is an R/R FL, an R/R transformed trFL, or an R/R DLBCL. In a particular embodiment, the B cell proliferative disorder is an R/R FL (e.g., a Grade 1, 2, or 3a, but not 3b, R/R FL according to the World Health Organization classification of lymphoid neoplasms (as referenced in Swerdlow S H, et al. Blood 2016; 127:2375-90)). In some embodiments, the R/R FL is R/R to at least two prior lines of therapy.

“Refractory disease” is defined as no complete remission to at least a first-line therapy. In one embodiment, refractory disease defined as no response to or relapse within 6 months of prior therapy. In one embodiment, refractory disease is characterized by one or more of the following: progressive disease (PD) as best response to first-line therapy, stable disease (SD) as best response after at least one first line therapy, or partial response (PR) as best response, and biopsy-proven residual disease or disease progression after the partial response. “Relapsed disease” is defined as complete remission to first-line therapy. In one embodiment, disease relapse is proven by biopsy. In one embodiment, subjects have relapsed after or failed to respond to at least one prior systemic treatment regimen or lines of therapy. In some embodiments, at least one of the prior lines of therapy comprises an anti-CD20 antibody (e.g., rituximab) and/or an alkylating agent (e.g., bendamustine, chlorambucil, cyclophosphamide, ifosfamide, mechlorethamine, melphalan, carmustine, lomustine, streptozocin, busulfan, dacarbazine, temozolomide, altretamine, or thiotepa).

As used herein, “treatment” (and grammatical variations thereof, such as “treat” or “treating”) refers to clinical intervention in an attempt to alter the natural course of the subject being treated, and can be performed either for prophylaxis or during the course of clinical pathology. Desirable effects of treatment include, but are not limited to, preventing recurrence of disease (e.g., R/R NHL, e.g., R/R FL, R/R trFL, or R/R DLBCL), alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies and antibody drug conjugates of the invention are used to delay development of a disease or to slow the progression of a disease.

“Delaying progression” of a disorder or disease means to defer, hinder, slow, retard, stabilize, and/or postpone development of the disease or disorder (e.g., an R/R NHL, e.g., R/R FL, R/R trFL, or R/R DLBCL). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

By “reduce” or “inhibit” is meant the ability to cause an overall decrease, for example, of 20%, 30%, 40%, 50%, 60%, 70%, 75%, 80%, 85%, 90%, 95%, or greater. For clarity the term includes also reduction to zero (or below the detection limit of the analytical method), i.e., complete abolishment or elimination. In certain embodiments, reduce or inhibit can refer to the reduction or inhibition of undesirable events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or central nervous system (CNS) toxicities, following treatment with mosunetuzumab using the step-up dosing regimen of the invention relative to unchanging, preset dosing with the target dose of mosunetuzumab. In other embodiments, reduce or inhibit can refer to effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP). In other embodiments reduce or inhibit can refer to the symptoms of the R/R NHL (e.g., R/R FL, (e.g., R/R FL, e.g., a Grade 1, 2, or 3a, but not 3b, R/R FL), R/R trFL, or R/R DLBCL) being treated, the presence or size of metastases, or the size of the primary tumor. In yet other embodiments, reducing or inhibiting cancer relapse means to reduce or inhibit tumor or cancer relapse, or tumor or cancer progression.

“Administering” means a method of giving a dosage of a compound (e.g., a bispecific antibody, e.g., mosunetuzumab) or a composition (e.g., a pharmaceutical composition, e.g., a pharmaceutical composition including a bispecific antibody, e.g., a composition or pharmaceutical composition including mosunetuzumab) to a subject. The compounds and/or compositions utilized in the methods (e.g., compounds and/or compositions containing mosunetuzumab) described herein can be administered intravenously (e.g., by intravenous infusion).

A “fixed” or “flat” dose of a therapeutic agent (e.g., a bispecific antibody, e.g., mosunetuzumab) herein refers to a dose that is administered to a subject without regard for the weight or body surface area (BSA) of the subject. The fixed or flat dose is therefore not provided as a mg/kg dose or a mg/m² dose, but rather as an absolute amount of the therapeutic agent (e.g., mg).

A “subject” or an “individual” is a mammal. Mammals include, but are not limited to, primates (e.g., humans and non-human primates such as monkeys), domesticated animals (e.g., cows, sheep, cats, dogs, and horses), rabbits, and rodents (e.g., mice and rats). In certain embodiments, the subject or individual is a human. In some embodiments, a subject is a patient.

A “reference population,” e.g., a “reference population of subjects,” as used herein, refers to a population (e.g., a population of subjects) that is used for comparison purposes. In some embodiments, a reference population of subjects comprises subjects having an R/R NHL (e.g., R/R FL, R/R trFL, or R/R DLBCL) and/or who have relapsed after and/or are refractory (R/R) to at least two prior lines of therapy, including where at least one prior line of therapy includes an anti-CD20 antibody and/or an alkylating agent (e.g., bendamustine, chlorambucil, cyclophosphamide, ifosfamide, mechlorethamine, melphalan, carmustine, lomustine, streptozocin, busulfan, dacarbazine, temozolomide, altretamine, or thiotepa). In some embodiments, each subject in the reference population of subjects is aged younger than about 65 years. Furthermore, one of skill in the art understands “reference population” in the context of the objective(s) of the comparisons being made. For example, in some embodiments, a reference population is a population of subjects aged younger than about 65 years, who are otherwise administered the same dosing regimen comprising mosunetuzumab as the subject or population of subjects who receive the dosing regimen comprising mosunetuzumab but is aged about 65 years or older.

“Individual response” or “response” can be assessed using any endpoint indicating a benefit to the subject, including, without limitation, (1) inhibition, to some extent, of disease progression (e.g., progression of an NHL (e.g., FL, e.g., R/R FL, e.g., a Grade 1, 2, or 3a, but not 3b, R/R FL according to the World Health Organization classification of lymphoid neoplasms (as referenced in Swerdlow S H, et al. Blood 2016; 127:2375-90)), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the NHL (e.g., FL, e.g., R/R FL); (6) increase or extend in the length of survival, including overall survival and progression-free survival; and/or (7) decreased mortality at a given point of time following treatment.

As used herein, “complete response” or “CR” refers to disappearance of all target lesions (i.e., all evidence of disease). In some embodiments, the “complete response rate” of a population of subjects is the rate of subjects in the population having a complete response.

As used herein, “partial response” or “PR” refers to at least a 30% decrease in the sum of the longest diameters (SLD) of target lesions, taking as reference the baseline SLD, or at least a 50% decrease in the product of the diameters (SPD) of target lesions, taking as reference the baseline SPD. In some embodiments, the “partial response rate” of a population of subjects is the rate of subjects in the population having a partial response rate.

As used herein, “objective response rate” (ORR) refers to the sum of complete response (CR) rate and partial response (PR) rate.

As used herein, “duration of objective response” or “duration of response” (DOR) is defined as the time from the initial occurrence of a documented objective response (e.g., a partial response or a complete response) until documented disease progression, or death due to any cause, whichever occurs first.

As used herein, “duration of complete response” (DoCR) is defined as the time from the initial occurrence of a documented complete response until documented disease progression, or death due to any cause, whichever occurs first.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

An “effective response” of a subject or a subject's “responsiveness” to treatment with a medicament and similar wording refers to the clinical or therapeutic benefit imparted to a subject as risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A subject who “does not have an effective response” to treatment refers to a subject who does not have any one of extending survival (including overall survival and progression free survival); objective response (including a complete response or a partial response); or improving signs or symptoms of cancer.

As used herein, “survival” refers to the subject remaining alive, and includes overall survival as well as progression-free survival.

As used herein, “overall survival” (OS) refers to the percentage of subjects in a group who are alive after a particular duration of time, e.g., 1 year or 5 years from the time of diagnosis or treatment.

As used herein, “progression-free survival” (PFS) refers to the length of time during and after treatment during which the disease being treated (e.g., NHL, e.g., FL (e.g., R/R FL, e.g., a Grade 1, 2, or 3a, but not 3b, R/R FL), R/R trFL, or R/R DLBCL) does not get worse. Progression-free survival may include the amount of time subjects have experienced a complete response or a partial response, as well as the amount of time subjects have experienced stable disease.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD, or at least a 50% increase in the SPD of target legions, taking as reference the smallest SPD, recorded since the treatment started or the presence of one or more new lesions.

As used herein, “delaying progression” of a disorder or disease means to defer, hinder, slow, retard, stabilize, and/or postpone development of the disease or disorder (e.g., NHL, e.g., FL (e.g., R/R FL, e.g., a Grade 1, 2, or 3a, but not 3b, R/R FL), R/R trFL, or R/R DLBCL). This delay can be of varying lengths of time, depending on the history of the disease and/or subject being treated. As is evident to one skilled in the art, a sufficient or significant delay can, in effect, encompass prevention, in that the subject does not develop the disease. For example, in a late-stage cancer, development of central nervous system (CNS) metastasis, may be delayed.

As used herein, the term “reducing or inhibiting cancer relapse” means to reduce or inhibit tumor or cancer relapse, or tumor or cancer progression.

By “extending survival” is meant increasing overall or progression-free survival in a treated subject relative to an untreated subject (e.g., relative to a subject not treated with the medicament), or relative to a subject who does not express a biomarker at the designated level, and/or relative to a subject treated with an approved anti-tumor agent. An objective response refers to a measurable response, including complete response (CR) or partial response (PR).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

The terms “full-length antibody,” “intact antibody,” and “whole antibody” are used herein interchangeably to refer to an antibody having a structure substantially similar to a native antibody structure or having heavy chains that contain an Fc region as defined herein.

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI Ref Seq No. NP_000724), which is 207 amino acids in length, and human CD3γ protein (NCBI Ref Seq No. NP_000064), which is 182 amino acids in length.

As used herein, the term “rituximab” or “RITUXAN®” refers to an anti-CD20 antibody (e.g., anti-CD20 monoclonal antibody) having the Proposed International Nonproprietary Names for Pharmaceutical Substances (Proposed INN) List 77 (WHO Drug Information, Vol. 11, No. 2, 1997, p. 99), or the CAS Registry Number 174722-31-7.

As used herein, the term “obinutuzumab” or “GAZYVA®” refers to an anti-CD20 antibody (e.g., anti-CD20 monoclonal antibody) having the Proposed International Nonproprietary Names for Pharmaceutical Substances (Proposed INN) List 99 (WHO Drug Information, Vol. 22, No. 2, 2008, p. 396), Proposed International Nonproprietary Names for Pharmaceutical Substances (Proposed INN) List 108 (WHO Drug Information, Vol. 26, No. 4, 2012, p. 453), or the CAS Registry Number 949142-50-1.

The term “cluster of differentiation 20” or “CD20,” as used herein, refers to any native CD20 from any vertebrate source, including mammals such as primates (e.g., humans) and rodents (e.g., mice and rats), unless otherwise indicated. The term encompasses “full-length,” unprocessed CD20, as well as any form of CD20 that results from processing in the cell. The term also encompasses naturally occurring variants of CD20, including, for example, splice variants or allelic variants. CD20 includes, for example, human CD20 protein (see, e.g., NCBI Ref Seq Nos. NP_068769.2 and NP_690605.1), which is 297 amino acids in length and may be generated, for example, from variant mRNA transcripts that lack a portion of the 5′ UTR (see, e.g., NCBI Ref Seq No. NM_021950.3) or longer variant mRNA transcripts (see, e.g., NCBI Ref Seq No. NM_152866.2).

The terms “anti-CD20/anti-CD3 bispecific antibody,” “bispecific anti-CD20/anti-CD3 antibody,” and “antibody that binds to CD20 and CD3,” or variants thereof, refer to mosunetuzumab.

As used herein, the term “mosunetuzumab” refers to an anti-CD20/anti-CD3 bispecific antibody having the International Nonproprietary Names for Pharmaceutical Substances (INN) List 117 (WHO Drug Information, Vol. 31, No. 2, 2017, p. 304-305), or the CAS Registry Number 1905409-39-3.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is ≤about 10% of the binding of the antibody to the target as measured, for example, by a radioimmunoassay (RIA). In certain embodiments, an antibody that specifically binds to a target has a dissociation constant (K_{D}) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. The term as used herein can be exhibited, for example, by a molecule having a K_{D} for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one embodiment, the term “specific binding” refers to binding where a molecule binds to a particular polypeptide or epitope on a particular polypeptide without substantially binding to any other polypeptide or polypeptide epitope.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the term “chemotherapeutic agent” refers to a compound useful in the treatment of a cancer, such as an NHL, e.g., (FL, e.g., R/R FL, e.g., a Grade 1, 2, or 3a, but not 3b, R/R FL, R/R trFL, or R/R DLBCL). Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, dacarbazine, mechlorethamine, cisplatin, methotrexate, 5-fluorouracil, and ara-C); and (x) pharmaceutically acceptable salts, acids, prodrugs, and derivatives of any of the above.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb, and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy)quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

The term “package insert” is used to refer to instructions customarily included in commercial packages of therapeutic products, that contain information about the indications, usage, dosage, administration, combination therapy, contraindications and/or warnings concerning the use of such therapeutic products.

### III. Therapeutic Methods

Provided herein are methods of treating a subject or a population of subjects having a relapsed and/or refractory (R/R) non-Hodgkin's lymphoma (NHL) (e.g., an R/R follicular lymphoma (FL), an R/R transformed FL (trFL), or an R/R diffuse large B cell lymphoma (DLBCL)) by intravenous administration (e.g., by intravenous infusion) of mosunetuzumab. In some embodiments, provided herein are methods of treating a subject or a population of subjects having an indolent NHL (e.g., a Grade 1, 2, or 3a, but not 3b FL) or an aggressive NHL (e.g., a DLBCL, a trFL, or a Grade 3b FL). In some instances, the subject or the population of subjects are relapsed and/or refractory (R/R) to two or more prior lines of therapy, while maintaining an acceptable safety profile (e.g., with respect to frequency and severity of adverse events, such as cytokine release syndrome (CRS)). In some instances, the two or more prior lines of therapy includes an anti-CD20 monoclonal antibody and/or an alkylating agent. In some instances, the subjects may be ineligible for autologous stem cell transplant (ASCT).

### A. Therapeutic Methods for Dosing of Mosunetuzumab

In one aspect, the invention provides a method of treating a subject or a population of subjects aged about 65 years or older (e.g., 65 years or older, 70 years or older, 75 years or older, 80 years or older, 85 years or older, 90 years or older, 95 years or older, or 100 years or older) having a relapsed or refractory (R/R) non-Hodgkin's lymphoma (NHL; e.g., R/R follicular lymphoma (FL), R/R transformed FL (trFL), or R/R diffuse large B cell lymphoma (DLBCL)) comprising intravenously administering to the subject mosunetuzumab in a dosing regimen comprising at least a first dosing cycle, a second dosing cycle, and a third dosing cycle, wherein: (a) the first three-dose dosing cycle comprises a first dose (C1D1), a second dose (C1D2), and a third dose (C1D3) of mosunetuzumab, wherein the C1 D1 is about 1 mg (e.g., 1 mg±0.01 mg, 0.025 mg, ±0.05 mg, ±0.075 mg, or ±0.1 mg; e.g., 1 mg), the C1 D2 is about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third dosing cycle comprises a single dose (C3D1) of mosunetuzumab, wherein the C3D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg). In some embodiments, the C1 D1 is 1±0.2 mg, the C1 D2 is 2±0.4 mg, the C1 D3 is 60±12 mg, the C2D1 is 60±12 mg, and the C3D1 is 30±6 mg.

In some instances, the first, second, and third dosing cycles are 21-day (±1 day) dosing cycles. In some instances, the method comprises administering the C1 D1, the C1 D2, and the C1 D3 on Days 1, 8 (±1 day), and 15 (±1 day), respectively, of the first dosing cycle. In some instances, the method comprises administering the C2D1 on Day 1 of the second dosing cycle. In some instances, the method comprises administering the C3D1 on Day 1 of the third dosing cycle.

In some instances, the dosing regimen further comprises one or more (e.g., one, two, three, four, five, six, seven, eight, nine, ten, 11, 12, 13, 14, or more) additional dosing cycles. In some instances, the dosing regimen comprises five to 14 (five, six, seven, eight, nine, ten, 11, 12, 13, or 14) additional dosing cycles. In a particular instance, the dosing regimen comprises five additional dosing cycles. In another particular instance, the dosing regimen comprises 14 additional dosing cycles. In some instances, the one or more (e.g., one, two, three, four, five, six, seven, eight, nine, ten, 11, 12, 13, 14, or more) additional dosing cycles are each 21-day (±1 day) dosing cycles.

In some instances, each of the one or more (e.g., one, two, three, four, five, six, seven, eight, nine, ten, 11, 12, 13, 14, or more) of the additional dosing cycles comprises an additional single dose of mosunetuzumab. In some instances, the additional single dose of mosunetuzumab is administered to the subject on Day 1 of each respective additional dosing cycle. In some instances, the additional single dose of mosunetuzumab is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg). In some embodiments, each additional dose of mosunetuzumab is 30±6 mg.

In one aspect, the invention provides a method of treating a population of subjects aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to each subject mosunetuzumab in a dosing regimen comprising at least a first 21-day (±1 day) dosing cycle, a second 21-day (±1 day) dosing cycle, and a third 21-day (±1 day) dosing cycle, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third dosing cycle comprises a single dose (C3D1) of mosunetuzumab administered on Day 1 of the third dosing cycle, wherein the C3D1 is about 30 mg (e.g., 30 mg±0.01 mg, ±0.025 mg, ±0.05 mg, 0.075 mg, ±0.1 mg, ±0.2 mg, ±0.3 mg, ±0.4 mg, ±0.5 mg, 0.75 mg, ±1 mg, ±1.5 mg, ±2 mg, or ±3 mg; e.g., 30 mg). In some embodiments, the C1 D1 is 1±0.2 mg, the C1 D2 is 2±0.4 mg, the C1 D3 is 60±12 mg, the C2D1 is 60±12 mg, and the C3D1 is 30±6 mg.

In one aspect, the invention provides a method of treating a population of subjects aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to each subject mosunetuzumab in a dosing regimen comprising eight 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third to eighth dosing cycles each comprises a single dose (C3D1-C8D1) of mosunetuzumab administered on Day 1 of each respective dosing cycle, wherein each of the C3D1-C8D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg). In some embodiments, the C1 D1 is 1±0.2 mg, the C1 D2 is 2±0.4 mg, the C1 D3 is 60±12 mg, the C2D1 is 60±12 mg, and each of the C3D1-C8D1 is 30±6 mg.

In one aspect, the invention provides a method of treating a population of subjects aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to each subject mosunetuzumab in a dosing regimen comprising 17 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third to 17th dosing cycles each comprises a single dose (C3D1-C17D1) of mosunetuzumab administered on Day 1 of each respective dosing cycle, wherein each of the C3D1-C17D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg). In some embodiments, the C1 D1 is 1±0.2 mg, the C1 D2 is 2±0.4 mg, the C1 D3 is 60±12 mg, the C2D1 is 60±12 mg, and each of the C3D1-C17D1 is 30±6 mg.

In some instances, the objective response rate in the population of subjects treated with the dosing regimens described herein is 65% (e.g., 70%, 75%, 80%, 85%, 90%, or 95%; e.g., 65-100%, 70-100%, 75-100%, 80-100%, 85-100%, 90-100%, 95-100%, 65-90%, 65-80%, 65-70%, 75-90%, 75-85%, 70-90%, or 80-90%; e.g., about 65%, about 70%, about 75%, about 80%, about 85%, about 90%, about 95%, or about 100%). In some instances, the objective response rate in the population of subjects is 75%. In some instances, the objective response rate in the population of subjects is 85%. In some instances, the objective response rate in the population of subjects is 85%.

In some instances, the complete response rate in the population of subjects treated with the dosing regimens described herein is 50% (e.g., ≥55%, ≥60%, ≥65%, ≥70%, ≥75%, ≥80%, ≥85%, ≥90%, or ≥95%; e.g., 50-100%, 60-100%, 70-100%, 80-100%, 90-100%, 95-100%, 50-90%, 50-80%, 50-70%, 50-60%, 50%-75%, 75-90%, 60-80%, 75-85%, 70-90%, or 80-90%; e.g., about 50%, about 55%, about 60%, about 65%, about 70%, about 75%, about 80%, about 85%, about 90%, about 95%, or about 100%). In some instances, the complete response rate in the population of subjects is ≥60%. In some instances, the complete response rate in the population of subjects is ≥70%.

In some instances, ≥40% (e.g., ≥45%, ≥50%, ≥55%, ≥60%, ≥65%, ≥70%, ≥75%, ≥80%, ≥85%, ≥90%, or ≥95%; e.g., 40-100%, 50-100%, 60-100%, 70-100%, 80-100%, 90-100%, 95-100%, 40-90%, 40-80%, 40-70%, 40-60%, 40-50%, 50%-70%, 50-60%, 75-90%, 60-80%, 75-85%, 70-90%, or 80-90%; e.g., about 40%, about 45%, about 50%, about 55%, about 60%, about 65%, about 70%, about 75%, about 80%, about 85%, about 90%, about 95%, or about 100%) of subjects in the population of subjects treated with the dosing regimens described herein having an objective response maintained remission for 18 months. In some instances, ≥50% of subjects having an objective response maintained remission for 18 months.

In some instances, ≥50% (e.g., ≥55%, ≥60%, ≥65%, ≥70%, ≥75%, ≥80%, ≥85%, ≥90%, or ≥95%; e.g., 50-100%, 60-100%, 70-100%, 80-100%, 90-100%, 95-100%, 50-90%, 50-80%, 50-70%, 50-60%, 50%-75%, 75-90%, 60-80%, 75-85%, 70-90%, or 80-90%; e.g., about 50%, about 55%, about 60%, about 65%, about 70%, about 75%, about 80%, about 85%, about 90%, about 95%, or about 100%) of subjects in the population of subjects treated with the dosing regimens described herein having a complete response maintained complete remission for 18 months. In some instances, ≥60% of subjects having a complete response maintained complete remission for 18 months.

In some instances, the median duration of response of subjects in the population of subjects treated with the dosing regimen is ≥9 months (e.g., ≥9, 10, 11, 12, 13, 14, 15, 18, 21, 24, 27, 30, 33, 36 months, or more; e.g., 9-12 months, 9-15 months, 9-18 months, 9-24 months, 12-18 months, 12-24 months, 12-36 months, 18-36 months, 24-36 months, or more; e.g., 10, 11, 12, 13, 14, 15, 16, 17, 18, 19, 20, 21, 24, 27, 30, 33, 36 months, or more). In some instances, the median duration of response in the population of subjects is ≥15 months. In some instances, the median duration of response in the population of subjects is ≥18 months.

In some instances, the median duration of complete response of subjects in the population of subjects treated with the dosing regimens described herein is ≥12 months (e.g., ≥9, 10, 11, 12, 13, 14, 15, 18, 21, 24, 27, 30, 33, 36 months, or more; e.g., 12-15 months, 12-18 months, 12-21 months, 12-24 months, 12-30 months, 12-36 months, 15-18 months, 15-24 months, 18-24 months, 24-36 months, 18-36 months, or more; e.g., 12, 13, 14, 15, 16, 17, 18, 19, 20, 21, 24, 27, 30, 33, 36 months, or more). In some instances, the median duration of complete response in the population of subjects is ≥16 months. In some instances, the median duration of complete response in the population of subjects is ≥18 months.

In some instances, ≥35% of subjects (e.g., ≥40%, ≥45%, ≥50%, ≥55%, ≥60%, ≥65%, ≥70%, ≥75%, ≥80%, ≥85%, ≥90%, or ≥95%; e.g., 35-100%, 40-100%, 50-100%, 60-100%, 70-100%, 80-100%, 90-100%, 95-100%, 35-90%, 35-80%, 35-70%, 35-60%, 35-50%, 35-45%, 40-80%, 40-60%, 50%-70%, 50-60%, 75-90%, 60-80%, 75-85%, 70-90%, or 80-90%; e.g., about 35%, about 40%, about 45%, about 50%, about 55%, about 60%, about 65%, about 70%, about 75%, about 80%, about 85%, about 90%, about 95%, or about 100%) in the population of subjects treated with the dosing regiments described herein have a progression-free survival of 18 months. In some instances, 45% of subjects have a progression-free survival of 18 months.

In some instances, the median progression-free survival in the population of subjects is ≥12 months (e.g., ≥9, 10, 11, 12, 13, 14, 15, 18, 21, 24, 27, 30, 33, 36 months, or more; e.g., 12-15 months, 12-18 months, 12-21 months, 12-24 months, 12-30 months, 12-36 months, 15-18 months, 15-24 months, 18-24 months, 24-36 months, 18-36 months, or more; e.g., 12, 13, 14, 15, 16, 17, 18, 19, 20, 21, 24, 27, 30, 33, 36 months, or more). In some instances, the median progression-free survival in the population of subjects is 17 months.

In one aspect, the invention provides a method of achieving an objective response, a complete response, or progression-free survival in a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL), wherein the method comprises intravenously administering to the subject mosunetuzumab in a dosing regimen comprising at least a first 21-day (±1 day) dosing cycle, a second 21-day (±1 day) dosing cycle, and a third 21-day (±1 day) dosing cycle, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, 0.075 mg, ±0.1 mg, ±0.2 mg, ±0.3 mg, +0.4 mg, ±0.5 mg, 0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third dosing cycle comprises a single dose (C3D1) of mosunetuzumab administered on Day 1 of the third dosing cycle, wherein the C3D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg). In some embodiments, the C1D1 is 1±0.2 mg, the C1D2 is 2±0.4 mg, the C1D3 is 60±12 mg, the C2D1 is 60±12 mg, and the C3D1 is 30±6 mg.

In one aspect, the invention provides a method of achieving an objective response, a complete response, or progression-free survival in a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL), wherein the method comprises intravenously administering to the subject mosunetuzumab in a dosing regimen comprising eight 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, 0.075 mg, or ±0.1 mg; e.g., 1 mg), the C1 D2 about 2 mg (e.g., 2 mg±0.01 mg, ±0.025 mg, ±0.05 mg, 0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, +2 mg, ±3 mg, ±4 mg, ±5 mg, ±6 mg; e.g., 60 mg); and (c) the third to eighth dosing cycles each comprises a single dose (C3D1-C8D1) of mosunetuzumab administered on Day 1 of each respective dosing cycle, wherein each of the C3D1-C8D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg). In some embodiments, the C1 D1 is 1±0.2 mg, the C1 D2 is 2±0.4 mg, the C1 D3 is 60±12 mg, the C2D1 is 60±12 mg, and each of the C3D1-C8D1 is 30±6 mg.

In one aspect, the invention provides a method of achieving an objective response, a complete response, or progression-free survival in a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL), wherein the method comprises intravenously administering to the subject mosunetuzumab in a dosing regimen comprising 17 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, 0.075 mg, or ±0.1 mg; e.g., 1 mg), the C1 D2 about 2 mg (e.g., 2 mg±0.01 mg, ±0.025 mg, ±0.05 mg, 0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, +2 mg, ±3 mg, ±4 mg, ±5 mg, ±6 mg; e.g., 60 mg); and (c) the third to 17th dosing cycles each comprises a single dose (C3D1-C17D1) of mosunetuzumab administered on Day 1 of each respective dosing cycle, wherein each of the C3D1-C17D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, 0.75 mg, ±1 mg, ±1.5 mg, ±2 mg, or ±3 mg; e.g., 30 mg). In some embodiments, the C1 D1 is 1±0.2 mg, the C1 D2 is 2±0.4 mg, the C1 D3 is 60±12 mg, the C2D1 is 60±12 mg, and each of the C3D1-C17D1 is 30±6 mg.

In some instances, the subject achieves an objective response. In some instances, the subject achieves a complete response. In some instances, the progression-free survival is maintained for 12 months (e.g., 12-15 months, 12-18 months, 12-21 months, 12-24 months, 12-30 months, 12-36 months, 15-18 months, 15-24 months, 18-24 months, 24-36 months, or 18-36 months; e.g., 12, 13, 14, 15, 16, 17, 18, 19, 20, 21, 24, 27, 30, 33, 36 months, or more). In some instances, the progression-free survival is maintained for 18 months.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to the subject mosunetuzumab in a dosing regimen comprising at least a first 21-day (±1 day) dosing cycle, a second 21-day (±1 day) dosing cycle, and a third 21-day (±1 day) dosing cycle, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, 0.075 mg, ±0.1 mg, ±0.2 mg, ±0.3 mg, +0.4 mg, ±0.5 mg, 0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third dosing cycle comprises a single dose (C3D1) of mosunetuzumab administered on Day 1 of the third dosing cycle, wherein the C3D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy. In some embodiments, the C1 D1 is 1±0.2 mg, the C1 D2 is 2±0.4 mg, the C1 D3 is 60±12 mg, the C2D1 is 60±12 mg, and the C3D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to the subject mosunetuzumab in a dosing regimen comprising eight 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, 0.075 mg, or ±0.1 mg; e.g., 1 mg), the C1 D2 about 2 mg (e.g., 2 mg±0.01 mg, ±0.025 mg, ±0.05 mg, 0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, +2 mg, ±3 mg, ±4 mg, ±5 mg, ±6 mg; e.g., 60 mg); and (c) the third to eighth dosing cycles each comprises a single dose (C3D1-C8D1) of mosunetuzumab administered on Day 1 of each respective dosing cycle, wherein each of the C3D1-C8D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy. In some embodiments, the C1 D1 is 1±0.2 mg, the C1 D2 is 2±0.4 mg, the C1 D3 is 60±12 mg, the C2D1 is 60±12 mg, and each of the C3D1-C8D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to the subject mosunetuzumab in a dosing regimen comprising 17 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, 0.075 mg, or ±0.1 mg; e.g., 1 mg), the C1 D2 about 2 mg (e.g., 2 mg±0.01 mg, ±0.025 mg, ±0.05 mg, 0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, +2 mg, ±3 mg, ±4 mg, ±5 mg, ±6 mg; e.g., 60 mg); and (c) the third to 17th dosing cycles each comprises a single dose (C3D1-C17D1) of mosunetuzumab administered on Day 1 of each respective dosing cycle, wherein each of the C3D1-C17D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, 0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy. In some embodiments, the C1 D1 is 1±0.2 mg, the C1 D2 is 2±0.4 mg, the C1 D3 is 60±12 mg, the C2D1 is 60±12 mg, and each of the C3D1-C17D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R FL comprising intravenously administering to the subject mosunetuzumab in a dosing regimen comprising at least a first 21-day (±1 day) dosing cycle, a second 21-day (±1 day) dosing cycle, and a third 21-day (±1 day) dosing cycle, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, +0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third dosing cycle comprises a single dose (C3D1) of mosunetuzumab administered on Day 1 of the third dosing cycle, wherein the C3D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy. In some embodiments, the C1 D1 is 1±0.2 mg, the C1 D2 is 2±0.4 mg, the C1 D3 is 60±12 mg, the C2D1 is 60±12 mg, and the C3D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R FL comprising intravenously administering to the subject mosunetuzumab in a dosing regimen comprising eight 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, 0.025 mg, ±0.05 mg, ±0.075 mg, ±0.1 mg, or ±0.2 mg; e.g., 2 mg), and the C1 D3 is about 60 mg (e.g., 60 mg±0.01 mg, 0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third to eighth dosing cycles each comprises a single dose (C3D1-C8D1) of mosunetuzumab administered on Day 1 of each respective dosing cycle, wherein each of the C3D1-C8D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy. In some embodiments, the C1 D1 is 1±0.2 mg, the C1 D2 is 2±0.4 mg, the C1 D3 is 60±12 mg, the C2D1 is 60±12 mg, and each of the C3D1-C8D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R FL comprising intravenously administering to the subject mosunetuzumab in a dosing regimen comprising 17 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, 0.025 mg, ±0.05 mg, ±0.075 mg, ±0.1 mg, or ±0.2 mg; e.g., 2 mg), and the C1 D3 is about 60 mg (e.g., 60 mg±0.01 mg, 0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third to 17th dosing cycles each comprises a single dose (C3D1-C17D1) of mosunetuzumab administered on Day 1 of each respective dosing cycle, wherein each of the C3D1-C17D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy. In some embodiments, the C1 D1 is 1±0.2 mg, the C1 D2 is 2±0.4 mg, the C1 D3 is 60±12 mg, the C2D1 is 60±12 mg, and each of the C3D1-C17D1 is 30±6 mg.

In some instances, the subject treated with the dosing regimen described herein and/or each subject in the population of subjects treated with the dosing regimen described herein has relapsed after, or is refractory to, two or more (e.g., two, three, four, five, six, seven, eight, nine, ten, 11, 12, or more) prior lines of therapy. In some instances, the two or more prior lines of therapy comprise an anti-CD20 monoclonal antibody (e.g., rituximab), an alkylating agent (e.g., bendamustine, chlorambucil, cyclophosphamide, ifosfamide, mechlorethamine, melphalan, carmustine, lomustine, streptozocin, busulfan, dacarbazine, temozolomide, altretamine, or thiotepa), or both an anti-CD20 monoclonal antibody and an alkylating agent.

In some instances, the R/R NHL described herein is R/R follicular lymphoma (FL), R/R transformed FL (trFL), or R/R diffuse large B cell lymphoma (DLBCL). In a particular instance, the R/R NHL is R/R FL. In a particular instance, the R/R FL is histologically documented as Grade 1, 2, or 3a, but not 3b according to the World Health Organization classification of lymphoid neoplasms (as referenced in Swerdlow S H, et al. Blood 2016; 127:2375-90).

In a particular instance, mosunetuzumab is administered to the subject treated with the dosing regimen described herein and/or to each subject in the population of subjects treated with the dosing regimen described herein by intravenous infusion.

In some instances, the subject described herein is a human subject. In some instances, the subjects described herein are human subjects.

In some instances, the median duration of progression-free survival of a population of subjects (i.e., elderly subjects) treated with a dosing regimen described herein is higher (e.g., by about 5%, 10%, 15%, 20%, 25%, 30%, 35%, 40%, 45%, 50%, or more) than a reference median duration of progression-free survival of a reference population of subjects treated with the same dosing regimen.

In some instances, the complete response rate in a population of subjects (i.e., elderly subjects) treated with a dosing regimen described herein is higher (e.g., by about 5%, 10%, 15%, 20%, 25%, 30%, 35%, 40%, 45%, 50%, or more) than a reference complete response rate in a reference population of subjects treated with the same dosing regimen.

In some instances, the objective response rate in a population of subjects (i.e., elderly subjects) treated with a dosing regimen described herein is higher (e.g., by about 5%, 10%, 15%, 20%, 25%, 30%, 35%, 40%, 45%, 50%, or more) than a reference objective response rate in a reference population of subjects treated with the same dosing regimen.

In some instances, the reference population of subjects does not comprise elderly subjects. In a particular instance, each subject in the reference population of subjects is aged about 18-64. In some instances, the reference population of subjects has R/R NHL (e.g., R/R FL, R/R trFL, or R/R DLBCL). In a particular instance, the R/R NHL is R/R FL. In a particular instance, the R/R FL is histologically documented as Grade 1, 2, or 3a, but not 3b according to the World Health Organization classification of lymphoid neoplasms (as referenced in Swerdlow S H, et al. Blood 2016; 127:2375-90). In some instances, each subject in the reference population of subjects has relapsed after, or is refractory to, two or more (e.g., two, three, four, five, six, seven, eight, nine, ten, 11, 12, or more) prior lines of therapy. In some instances, the two or more prior lines of therapy comprise an anti-CD20 monoclonal antibody (e.g., rituximab), an alkylating agent (e.g., bendamustine, chlorambucil, cyclophosphamide, ifosfamide, mechlorethamine, melphalan, carmustine, lomustine, streptozocin, busulfan, dacarbazine, temozolomide, altretamine, or thiotepa), or both an anti-CD20 monoclonal antibody and an alkylating agent.

### B. Dosing Strategies for Mitigating Adverse Events

The present invention relates to methods of treating a subject or a population of subjects aged about 65 years or older and having an R/R NHL (e.g., an R/R FL, R/R DLBCL, or R/R trFL), by intravenous administration of mosunetuzumab, while maintaining an acceptable safety profile (e.g., with respect to frequency and severity of adverse events (AEs; e.g., including serious adverse events (SAEs)), such as cytokine release syndrome (CRS). In some instances, the subject or the population of subjects are relapsed and/or refractory (R/R) to two or more prior lines of therapy, while maintaining an acceptable safety profile (e.g., with respect to frequency and severity of adverse events, such as cytokine release syndrome (CRS)). In some instances, the two or more prior lines of therapy includes an anti-CD20 monoclonal antibody and/or an alkylating agent. In some instances, the subjects may be ineligible for autologous stem cell transplant (ASCT).

1. CRS Symptoms and Grading

Any of the methods described herein may involve monitoring a subject for cytokine release syndrome (CRS), e.g., a CRS event following commencement of any of the methods described above. Current clinical management focuses on treating the individual signs and symptoms, providing supportive care, and attempting to dampen the inflammatory response using a high dose of corticosteroids. However, this approach is not always successful, especially in the case of late intervention. The CRS grading criteria used by the methods described herein are published by the American Society for Transplantation and Cellular Therapy (ASTCT) to define mild, moderate, severe, or life-threatening CRS and harmonize reporting across clinical trials to allow rapid recognition and treatment of CRS (Lee et al. Biol Blood Marrow Transplantation. 25(4): 625-638, 2019). The ASTCT criteria is intended to be objective, easy to apply, and more accurately categorize the severity of CRS. This CRS grading system is shown below in Table 1.

TABLE 1
CRS Grading System

| CRS | | | | |
|---|---|---|---|---|
| Parameter | Grade 1 | Grade 2 | Grade 3 | Grade 4 |
| Fever | Temperature | Temperature | Temperature | Temperature |
| | ≥38° C. | ≥38° C. | ≥38° C. | ≥38° C. |
| | | with | | |
| Hypotension | None | Not requiring | Requiring a | Requiring multiple |
| | | vasopressors | vasopressor with or | vasopressors |
| | | | without vasopressin | (excluding |
| | | | | vasopressin) |
| | | and/or | | |
| Hypoxia | None | Requiring low-glow | Requiring high-flow | Requiring positive |
| | | nasal cannula or | nasal cannula, | pressure (e.g., |
| | | blow-by | facemask, | CPAP, BiPAP |
| | | | nonrebreather mask | intubation and |
| | | | or Venturi mask | mechanical |
| | | | | ventilation) |

ASTCT = American Society for Transplantation and Cellular Therapy; BiPAP = bilevel positive airway pressure; CPAP = continuous positive airway pressure; CRS = cytokine release syndrome; CTCAE = Common Terminology Criteria for Adverse Events.

Fever is defined as a temperature 38° C. not attributable to any other cause. In subjects who have CRS then receive antipyretic or anti-cytokine therapy such as tocilizumab or steroids, fever is no longer required to grade subsequent CRS severity. In this case, CRS grading is determined by hypotension and/or hypoxia.

CRS grade is determined by the more severe event, hypotension or hypoxia not attributable to any other cause. For example, a subject with temperature of 39.5° C., hypotension requiring 1 vasopressor, and hypoxia requiring low-flow nasal cannula is classified as Grade 3 CRS.

Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

CRS is associated with elevations in a wide array of cytokines, including marked elevations in IFN-γ, IL-6, and TNF-α levels. Emerging evidence implicates IL-6, in particular, as a central mediator in CRS. IL-6 is a proinflammatory, multi-functional cytokine produced by a variety of cell types, which has been shown to be involved in a diverse array of physiological processes, including T cell activation. Regardless of the inciting agent, CRS is associated with high IL-6 levels (Nagorsen et al. Cytokine. 25(1): 31-5, 2004; Lee et al. Blood. 124(2): 188-95, 2014); Doesegger et al. Clin. Transl. Immunology. 4(7): e39, 2015), and IL-6 correlates with the severity of CRS, with subjects who experience a Grade 4 or 5 CRS event having much higher IL-6 levels compared to subjects who do not experience CRS or experience milder CRS (Grade 0-3) (Chen et al. J. Immunol. Methods. 434:1-8, 2016).

Therefore, blocking the inflammatory action of IL-6 using an agent that inhibits IL-6-mediated signaling to manage CRS observed in subjects during the double-step fractionated, dose-escalation dosing regimen is an alternative to steroid treatment that would not be expected to negatively impact T cell function or diminish the efficacy or clinical benefit of mosunetuzumab therapy in the treatment of CD20-positive cell proliferative disorders (e.g., a B cell proliferative disorders).

If the subject has a CRS event that does not resolve or worsens within 24 hours of administering the IL-6R antagonist to treat the symptoms of the CRS event, and the method may further comprise administering to the subject one or more additional doses of the IL-6R antagonist to manage the CRS event. The subject may be administered a corticosteroid, such as methylprednisolone or dexamethasone if CRS event is not managed through administration of the IL-6R antagonist.

2. Other Adverse Events and Grading

Any of the methods described herein may involve monitoring a subject for additional non-CRS adverse events. Incidence, nature, and severity of physical findings and adverse events, with severity determined according to the National Cancer Institute Common Terminology Criteria for Adverse Events version 5 (NCI CTCAE v5.0; cancer.gov).

3. Dosing Regimens with Acceptable Safety Profiles

The present invention relates to methods of treating a subject or a population of subjects aged about 65 years or older and having an R/R NHL (e.g., an R/R FL, R/R DLBCL, or R/R trFL), by intravenous administration of mosunetuzumab, while maintaining an acceptable safety profile. In particular, the present invention features dosing regimens including mosunetuzumab and one or more additional therapeutic agents useful for maintaining an acceptable safety profile and/or reducing the risk of an adverse event (e.g., a cytokine release syndrome (CRS) event). In some instances, the dosing regimen includes premedication treatment, wherein one or more additional therapeutic agents is administered prior to administering any dose of mosunetuzumab (e.g., prior to administering the C1 D1, C1 D2, C1 D3, or any of the C2D1-C17D1 doses of mosunetuzumab). In some instances, the dosing regimens described herein include premedication with a corticosteroid, an anti-histamine, and/or an anti-pyretic. In a particular instance, the dosing regimens described herein include premedication with a corticosteroid, an anti-histamine, and an anti-pyretic. In another particular instance, the dosing regimens described herein include premedication with a corticosteroid. In other instances, a subject is administered one or more additional therapeutic agents after experiencing an adverse event (e.g., a CRS event). In some instances, a subject is administered a corticosteroid or an IL-6R antagonist (e.g., tocilizumab) after experiencing an adverse event (e.g., a CRS event). In a particular instance, a subject is administered tocilizumab after experiencing a CRS event.

In some instances, the method further comprises administering to the subject or to each subject in the population of subjects one or more (e.g., one, two, three, four, five, six, or more) additional therapeutic agents.

In some instances, the one or more (e.g., one, two, three, four, five, six, or more) additional therapeutic agents comprises tocilizumab.

In some instances, the one or more (e.g., one, two, three, four, five, six, or more) additional therapeutic agents comprises a corticosteroid. In some instances, the corticosteroid comprises dexamethasone or methylprednisolone. In some instances, the corticosteroid is administered to the subject or to each subject in the population of subjects at least one hour (e.g., 60 70, 80, 90, 120, 150, 180, 210, 240, 300 minutes, or more) prior to the administration of any dose of mosunetuzumab. In some instances, the corticosteroid is only administered during dosing cycle 1 or dosing cycle 2. In some instances, the corticosteroid is additionally administered during one or more dosing cycles after dosing cycle 2 (e.g., in dosing cycle 3, 4, 5, 6, 7, 8, 9, 10, 11, 12, 13, 14, 15, 16, and/or 17). In some instances, the corticosteroid is administered intravenously. In some instances, the corticosteroid comprises dexamethasone and is administered at a dose of about 20 mg (e.g., 20 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, or ±2 mg; e.g., 20 mg). In some instances, the corticosteroid comprises methylprednisolone and is administered at a dose of about 80 mg (e.g., 80 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±8 mg, ±7 mg, or ±8 mg; e.g., 80 mg).

In some instances, the one or more (e.g., one, two, three, four, five, six, or more) additional therapeutic agents comprises an anti-histamine. In some instances, the anti-histamine is administered to the subject or to each subject in the population of subjects at least 30 minutes (e.g., 30, 35, 40, 45, 50, 60 70, 80, 90, 120, 150, 180 minutes, or more) prior to the administration of any dose of mosunetuzumab. In some instances, the anti-histamine is only administered during dosing cycle 1 or dosing cycle 2. In some instances, the anti-histamine is additionally administered during one or more dosing cycles after dosing cycle 2 (e.g., in dosing cycle 3, 4, 5, 6, 7, 8, 9, 10, 11, 12, 13, 14, 15, 16, and/or 17). In some instances, the anti-histamine is administered orally or intravenously. In some instances, the anti-histamine comprises diphenhydramine hydrochloride and is administered at a dose of about 50-100 mg (e.g., 50-90 mg, 50-80 mg, 50-70 mg, 50-60 mg, 60-100 mg, 70-100 mg, 80-100 mg, 90-100 mg, 70-80 mg, 60-90 mg, 60-80 mg, 70-90 mg, or 65-85 mg; e.g., about 50 mg, about 55 mg, about 60 mg, about 65 mg, about 70 mg, about 75 mg, about 80 mg, about 85 mg, about 90 mg, about 95 mg, or about 100 mg).

In some instances, the one or more (e.g., one, two, three, four, five, six, or more) additional therapeutic agents comprises an anti-pyretic. In some instances, the anti-pyretic is administered to the subject or to each subject in the population of subjects at least 30 minutes (e.g., 30, 35, 40, 45, 50, 60 70, 80, 90, 120, 150, 180 minutes, or more) prior to the administration of any dose of mosunetuzumab. In some instances, the anti-pyretic is only administered during dosing cycle 1 or dosing cycle 2. In some instances, the anti-pyretic is additionally administered during one or more dosing cycles after dosing cycle 2 (e.g., in dosing cycle 3, 4, 5, 6, 7, 8, 9, 10, 11, 12, 13, 14, 15, 16, and/or 17). In some instances, the anti-pyretic is administered orally. In some instances, the anti-pyretic comprises acetaminophen and is administered at a dose of about 500-1000 mg (e.g., 500-900 mg, 500-800 mg, 500-700 mg, 500-600 mg, 600-1000 mg, 700-1000 mg, 800-1000 mg, 900-1000 mg, 700-800 mg, 600-900 mg, 600-800 mg, 700-900 mg, or 650-850 mg; e.g., about 500 mg, about 550 mg, about 600 mg, about 650 mg, about 700 mg, about 750 mg, about 800 mg, about 850 mg, about 900 mg, about 950 mg, or about 1000 mg).

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to the subject mosunetuzumab in a dosing regimen comprising at least a first 21-day (±1 day) dosing cycle, a second 21-day (±1 day) dosing cycle, and a third 21-day (±1 day) dosing cycle, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, 0.075 mg, ±0.1 mg, ±0.2 mg, ±0.3 mg, +0.4 mg, ±0.5 mg, 0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third dosing cycle comprises a single dose (C3D1) of mosunetuzumab administered on Day 1 of the third dosing cycle, wherein the C3D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy, and wherein the dosing regimen further comprises: (i) administering a corticosteroid, wherein a single dose of the corticosteroid is administered to the subject at least one hour (e.g., 60 70, 80, 90, 120, 150, 180, 210, 240, 300 minutes, or more) prior to the administration of each of the C1 D1, C1 D2, C1 D3, and C2D1 of mosunetuzumab; (ii) administering an anti-histamine, wherein a single dose of the anti-histamine is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab; and/or (iii) administering an anti-pyretic, wherein a single dose of the anti-pyretic is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab. In some embodiments, the C1 D1 is 1±0.2 mg, the C1 D2 is 2±0.4 mg, the C1 D3 is 60±12 mg, the C2D1 is 60±12 mg, and the C3D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to the subject mosunetuzumab in a dosing regimen comprising eight 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, 0.075 mg, or ±0.1 mg; e.g., 1 mg), the C1 D2 about 2 mg (e.g., 2 mg±0.01 mg, ±0.025 mg, ±0.05 mg, 0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, +2 mg, ±3 mg, ±4 mg, ±5 mg, ±6 mg; e.g., 60 mg); and (c) the third to eighth dosing cycles each comprises a single dose (C3D1-C8D1) of mosunetuzumab administered on Day 1 of each respective dosing cycle, wherein each of the C3D1-C8D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more prior (e.g., two, three, four, five, six, seven, eight, nine, ten, 11, 12, or more) lines of therapy, and wherein the dosing regimen further comprises: (i) administering a corticosteroid, wherein a single dose of the corticosteroid is administered to the subject at least one hour (e.g., 60 70, 80, 90, 120, 150, 180, 210, 240, 300 minutes, or more) prior to the administration of each of the C1 D1, C1 D2, C1 D3, and C2D1 of mosunetuzumab; (ii) administering an anti-histamine, wherein a single dose of the anti-histamine is administered to the subject at least 30 (e.g., 30, 35, 40, 45, 50, 60 70, 80, 90, 120, 150, 180 minutes, or more) minutes prior to the administration of each of the C1 D1, C1 D2, C1 D3, and C2D1 of mosunetuzumab; and/or (iii) administering an anti-pyretic, wherein a single dose of the anti-pyretic is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab. In some embodiments, the C1 D1 is 1±0.2 mg, the C1D2 is 2±0.4 mg, the C1D3 is 60±12 mg, the C2D1 is 60±12 mg, and each of the C3D1-C8D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R NHL (e.g., R/R FL, R/R DLBCL, or R/R trFL) comprising intravenously administering to the subject mosunetuzumab in a dosing regimen comprising 17 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, 0.075 mg, or ±0.1 mg; e.g., 1 mg), the C1 D2 about 2 mg (e.g., 2 mg±0.01 mg, ±0.025 mg, ±0.05 mg, 0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, +2 mg, ±3 mg, ±4 mg, ±5 mg, ±6 mg; e.g., 60 mg); and (c) the third to 17th dosing cycles each comprises a single dose (C3D1-C17D1) of mosunetuzumab administered on Day 1 of each respective dosing cycle, wherein each of the C3D1-C17D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, 0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy, and wherein the dosing regimen further comprises: (i) administering a corticosteroid, wherein a single dose of the corticosteroid is administered to the subject at least one hour (e.g., 60 70, 80, 90, 120, 150, 180, 210, 240, 300 minutes, or more) prior to the administration of each of the C1 D1, C1 D2, C1 D3, and C2D1 of mosunetuzumab; (ii) administering an anti-histamine, wherein a single dose of the anti-histamine is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab; and/or (iii) administering an anti-pyretic, wherein a single dose of the anti-pyretic is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab. In some embodiments, the C1 D1 is 1±0.2 mg, the C1 D2 is 2±0.4 mg, the C1 D3 is 60±12 mg, the C2D1 is 60±12 mg, and each of the C3D1-C17D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R FL comprising intravenously administering to the subject mosunetuzumab in a dosing regimen comprising at least a first 21-day (±1 day) dosing cycle, a second 21-day (±1 day) dosing cycle, and a third 21-day (±1 day) dosing cycle, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, +0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third dosing cycle comprises a single dose (C3D1) of mosunetuzumab administered on Day 1 of the third dosing cycle, wherein the C3D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy, and wherein the dosing regimen further comprises: (i) administering a corticosteroid, wherein a single dose of the corticosteroid is administered to the subject at least one hour (e.g., 60 70, 80, 90, 120, 150, 180, 210, 240, 300 minutes, or more) prior to the administration of each of the C1 D1, C1 D2, C1 D3, and C2D1 of mosunetuzumab; (ii) administering an anti-histamine, wherein a single dose of the anti-histamine is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab; and/or (iii) administering an anti-pyretic, wherein a single dose of the anti-pyretic is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab. In some embodiments, the C1 D1 is 1±0.2 mg, the C1 D2 is 2±0.4 mg, the C1 D3 is 60±12 mg, the C2D1 is 60±12 mg, and the C3D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R FL comprising intravenously administering to the subject mosunetuzumab in a dosing regimen comprising eight 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, ±0.025 mg, ±0.05 mg, ±0.075 mg, ±0.1 mg, or ±0.2 mg; e.g., 2 mg), and the C1 D3 is about 60 mg (e.g., 60 mg±0.01 mg, 0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third to eighth dosing cycles each comprises a single dose (C3D1-C8D1) of mosunetuzumab administered on Day 1 of each respective dosing cycle, wherein each of the C3D1-C8D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more prior (e.g., two, three, four, five, six, seven, eight, nine, ten, 11, 12, or more) lines of therapy, and wherein the dosing regimen further comprises: (i) administering a corticosteroid, wherein a single dose of the corticosteroid is administered to the subject at least one hour (e.g., 60 70, 80, 90, 120, 150, 180, 210, 240, 300 minutes, or more) prior to the administration of each of the C1 D1, C1 D2, C1 D3, and C2D1 of mosunetuzumab; (ii) administering an anti-histamine, wherein a single dose of the anti-histamine is administered to the subject at least 30 (e.g., 30, 35, 40, 45, 50, 60 70, 80, 90, 120, 150, 180 minutes, or more) minutes prior to the administration of each of the C1 D1, C1 D2, C1 D3, and C2D1 of mosunetuzumab; and/or (iii) administering an anti-pyretic, wherein a single dose of the anti-pyretic is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab. In some embodiments, the C1 D1 is 1±0.2 mg, the C1 D2 is 2±0.4 mg, the C1 D3 is 60±12 mg, the C2D1 is 60±12 mg, and each of the C3D1-C8D1 is 30±6 mg.

In one aspect, the invention provides a method of treating a subject aged about 65 years or older (e.g., 65 years or older, 70 years or older, 75 years or older, 80 years or older, 85 years or older, 90 years or older, 95 years or older, or 100 years or older) having an R/R FL comprising intravenously administering to the subject mosunetuzumab in a dosing regimen comprising 17 21-day (±1 day) dosing cycles, wherein: (a) the first three-dose dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1 D1 is about 1 mg (e.g., 1 mg±0.01 mg, ±0.025 mg, ±0.05 mg, ±0.075 mg, or ±0.1 mg; e.g., 1 mg), the C1 D2 about 2 mg (e.g., 2 mg±0.01 mg, 0.025 mg, ±0.05 mg, ±0.075 mg, ±0.1 mg, or ±0.2 mg; e.g., 2 mg), and the C1 D3 is about 60 mg (e.g., 60 mg±0.01 mg, 0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); (b) the second dosing cycle comprises a single dose (C2D1) of mosunetuzumab administered on Day 1 of the second dosing cycle, wherein the C2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg; e.g., 60 mg); and (c) the third to 17th dosing cycles each comprises a single dose (C3D1-C17D1) of mosunetuzumab administered on Day 1 of each respective dosing cycle, wherein each of the C3D1-C17D1 is about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg), wherein the subject has relapsed after, or is refractory to, two or more (e.g., two, three, four, five, six, seven, eight, nine, ten, 11, 12, or more) prior lines of therapy, and wherein the dosing regimen further comprises: (i) administering a corticosteroid, wherein a single dose of the corticosteroid is administered to the subject at least one hour (e.g., 60 70, 80, 90, 120, 150, 180, 210, 240, 300 minutes, or more) prior to the administration of each of the C1 D1, C1 D2, C1 D3, and C2D1 of mosunetuzumab; (ii) administering an anti-histamine, wherein a single dose of the anti-histamine is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab; and/or (iii) administering an anti-pyretic, wherein a single dose of the anti-pyretic is administered to the subject at least 30 minutes (e.g., 30, 35, 40, 45, 50, 60 70, 80, 90, 120, 150, 180 minutes, or more) prior to the administration of each of the C1 D1, C1 D2, C1 D3, and C2D1 of mosunetuzumab. In some embodiments, the C1 D1 is 1±0.2 mg, the C1D2 is 2±0.4 mg, the C1D3 is 60±12 mg, the C2D1 is 60±12 mg, and each of the C3D1-C17D1 is 30±6 mg.

In some instances, the subject has relapsed after, or is refractory to, two or more (e.g., two, three, four, five, six, seven, eight, nine, ten, 11, 12, or more) prior lines of therapy. In some instances, the two or more prior lines of therapy comprise an anti-CD20 monoclonal antibody (e.g., rituximab), an alkylating agent (e.g., bendamustine, chlorambucil, cyclophosphamide, ifosfamide, mechlorethamine, melphalan, carmustine, lomustine, streptozocin, busulfan, dacarbazine, temozolomide, altretamine, or thiotepa), or both an anti-CD20 monoclonal antibody and an alkylating agent.

In some instances, the R/R NHL is R/R follicular lymphoma (FL), R/R transformed FL (trFL), or R/R diffuse large B cell lymphoma (DLBCL). In a particular instance, the R/R NHL is R/R FL. In a particular instance, the R/R FL is histologically documented as Grade 1, 2, or 3a, but not 3b according to the World Health Organization classification of lymphoid neoplasms (as referenced in Swerdlow S H, et al. Blood 2016; 127:2375-90).

In a particular instance, mosunetuzumab is administered by intravenous infusion.

In some instances, (i) a single dose of the corticosteroid is administered prior to the administration of any dose of mosunetuzumab in any of the one or more of any of the dosing cycles after the second dosing cycle (e.g., prior to administration of the C3D1, C4D1, C5D1, C6D1, C7D1, C8D1, C9D1, C10D1, C11 D1, C12D1, C13D1, and/or C14D1 of mosunetuzumab); (ii) a single dose of the anti-histamine is administered prior to the administration of any dose of mosunetuzumab in any of the one or more of any of the dosing cycles after the second dosing cycle (e.g., prior to administration of the C3D1, C4D1, C5D1, C6D1, C7D1, C8D1, C9D1, C10D1, C11D1, C12D1, C13D1, and/or C14D1 of mosunetuzumab); and/or (iii) a single dose of the anti-pyretic is administered prior to the administration of any dose of mosunetuzumab in any of the one or more of any of the dosing cycles after the second dosing cycle (e.g., prior to administration of the C3D1, C4D1, C5D1, C6D1, C7D1, C8D1, C9D1, C10D1, C11 D1, C12D1, C13D1, and/or C14D1 of mosunetuzumab).

In some instances, (i) the corticosteroid is administered intravenously; (ii) the anti-histamine is administered orally or intravenously; and/or (iii) the anti-pyretic is administered orally.

In some instances, (i) the corticosteroid comprises dexamethasone and is administered at a dose of about 20 mg (e.g., 20 mg±0.01 mg, 0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, or ±2 mg; e.g., 20 mg) or the corticosteroid comprises methylprednisolone and is administered at a dose of about 80 mg (e.g., 80 mg±0.01 mg, ±0.025 mg, ±0.05 mg, ±0.075 mg, ±0.1 mg, ±0.2 mg, ±0.3 mg, ±0.4 mg, ±0.5 mg, 0.75 mg, ±1 mg, ±1.5 mg, ±2 mg, ±3 mg, ±4 mg, ±5 mg, ±8 mg, ±7 mg, or ±8 mg; e.g., 80 mg); (ii) the anti-histamine comprises diphenhydramine hydrochloride and is administered at a dose of about 50-100 mg (e.g., 50-90 mg, 50-80 mg, 50-70 mg, 50-60 mg, 60-100 mg, 70-100 mg, 80-100 mg, 90-100 mg, 70-80 mg, 60-90 mg, 60-80 mg, 70-90 mg, or 65-85 mg; e.g., about 50 mg, about 55 mg, about 60 mg, about 65 mg, about 70 mg, about 75 mg, about 80 mg, about 85 mg, about 90 mg, about 95 mg, or about 100 mg); and/or (iii) the anti-pyretic is acetaminophen and is administered at a dose of about 500-1000 mg (e.g., 500-900 mg, 500-800 mg, 500-700 mg, 500-600 mg, 600-1000 mg, 700-1000 mg, 800-1000 mg, 900-1000 mg, 700-800 mg, 600-900 mg, 600-800 mg, 700-900 mg, or 650-850 mg; e.g., about 500 mg, about 550 mg, about 600 mg, about 650 mg, about 700 mg, about 750 mg, about 800 mg, about 850 mg, about 900 mg, about 950 mg, or about 1000 mg).

In some instances, the rate of serious adverse events, excluding Grade 5 malignant neoplasm progression, in a population of subjects (i.e., elderly subjects) treated with a dosing regimen described herein is ≤45% (e.g., ≤40%, 35%, 30%, 25%, 20%, 15%, 10%, or 5%; e.g., 0-45%, 0-35%, 0-25%, 0-15%, 0-10%, 0-5%, 5-15%, 15-30%, 30-45%, 10-30%, 20-40%, 35-45%, 30-40%, 20-30%, or 10-20%; e.g., about 0%, about 5%, about 10%, about 15%, about 20%, about 25%, about 30%, about 35%, about 40% or about 45%). In some instances, the rate of serious adverse events, excluding Grade 5 malignant neoplasm progression, in the population of subjects is ≤40%. In some instances, the rate of serious adverse events, excluding Grade 5 malignant neoplasm progression, in the population of subjects is ≤50% (e.g., about 50%, about 49%, about 48%, about 47%, or about 46%).

In some embodiments, the rate of serious adverse events related to mosunetuzumab in the population of subjects is ≤40% (e.g., ≤35%, 30%, 25%, 20%, 15%, 10%, or 5%; e.g., 0-40%, 0-35%, 0-25%, 0-15%, 0-10%, 0-5%, 5-15%, 15-30%, 30-40%, 10-30%, 20-40%, 30-40%, 20-30%, or 10-20%; e.g., about 0%, about 5%, about 10%, about 15%, about 20%, about 25%, about 30%, about 35%, or about 40%). In some embodiments, the rate of serious adverse events related to mosunetuzumab in the population of subjects is ≤30%. In some instances, the rate of serious adverse events related to mosunetuzumab in the population of subjects is ≤35%.

In some instances, the rate of cytokine release syndrome having a grade of 3 or higher (as defined by the American Society for Transplantation and Cellular Therapy, 2018; ASTCT) in a population of subjects (i.e., elderly subjects) treated with a dosing regimen described herein is ≤5% (e.g., ≤4%, 3%, 2%, or 1%; e.g., 0-5%, 1-5%, 2-5%, 3-5%, 4-5%, 1-3%, 2-4%, or 0-3%; e.g., about 0%, about 1%, about 2%, about 3%, about 4%, or about 5%). In some instances, the rate of cytokine release syndrome having a grade of 3 or higher (as defined by the ASTCT) in the population of subjects is ≤3%.

In some instances, the rate of cytokine release syndrome of any grade (as defined by the ASTCT) in a population of subjects (i.e., elderly subjects) treated with a dosing regimen described herein is ≤40% (e.g., ≤35%, 30%, 25%, 20%, 15%, 10%, or 5%; e.g., 0-40%, 0-35%, 0-25%, 0-15%, 0-10%, 0-5%, 5-15%, 15-30%, 25-40%, 10-30%, 20-40%, 25-35%, 35-40%, 30-40%, 20-30%, or 10-20%; e.g., about 0%, about 5%, about 10%, about 15%, about 20%, about 25%, about 30%, about 35%, or about 40%). In some instances, the rate of cytokine release syndrome of any grade (as defined by the ASTCT) in the population of subjects is ≤30%. In some embodiments, the rate of cytokine release syndrome of any grade (as defined by the ASTCT) in the population of subjects is ≤50% (e.g., ≤45%; e.g., 40-50%, 30-50%, 25-50%, 20-50%, 10-50%, 0-50%, 40-45%, 30-35%, 20-45%, 10-45%, 0-45%; e.g., about 41%, about 42%, about 43%, about 44%, about 45%, about 46%, about 47%, about 48%, about 49%, or about 50%).

In some instances, the rate of serious adverse events (SAEs), excluding Grade 5 malignant neoplasm progression, of a population of subjects (i.e., elderly subjects) treated with a dosing regimen described herein is lower (e.g., by about 5%, 10%, 15%, 20%, 25%, 30%, 35%, 40%, 45%, 50%, or more) than a reference rate of SAEs, excluding Grade 5 malignant neoplasm progression, of a reference population of subjects treated with the same dosing regimen.

In some instances, the rate of serious adverse events (SAEs) related to mosunetuzumab of a population of subjects (i.e., elderly subjects) treated with a dosing regimen described herein is lower (e.g., by about 5%, 10%, 15%, 20%, 25%, 30%, 35%, 40%, 45%, 50%, or more) than a reference rate of SAEs related to mosunetuzumab of a reference population of subjects treated with the same dosing regimen.

In some instances, the rate of cytokine release syndrome (CRS) events of a population of subjects (i.e., elderly subjects) treated with a dosing regimen described herein is lower (e.g., by about 5%, 10%, 15%, 20%, 25%, 30%, 35%, 40%, 45%, 50%, or more) than a reference rate of CRS events of a reference population of subjects treated with the same dosing regimen.

In some instances, the reference population of subjects does not comprise elderly subjects. In a particular instance, each subject in the reference population of subjects is aged about 18-64. In some instances, the reference population of subjects has R/R NHL (e.g., R/R FL, R/R trFL, or R/R DLBCL). In a particular instance, the R/R NHL is R/R FL. In a particular instance, the R/R FL is histologically documented as Grade 1, 2, or 3a, but not 3b according to the World Health Organization classification of lymphoid neoplasms (as referenced in Swerdlow S H, et al. Blood 2016; 127:2375-90). In some instances, each subject in the reference population of subjects has relapsed after, or is refractory to, two or more (e.g., two, three, four, five, six, seven, eight, nine, ten, 11, 12, or more) prior lines of therapy. In some instances, the two or more prior lines of therapy comprise an anti-CD20 monoclonal antibody (e.g., rituximab), an alkylating agent (e.g., bendamustine, chlorambucil, cyclophosphamide, ifosfamide, mechlorethamine, melphalan, carmustine, lomustine, streptozocin, busulfan, dacarbazine, temozolomide, altretamine, or thiotepa), or both an anti-CD20 monoclonal antibody and an alkylating agent.

In some instances, the one or more additional therapeutic agents is a corticosteroid or an IL-6R antagonist, and is used to treat subjects that experience cytokine release syndrome (CRS). In some instances, the one or more additional therapeutic agents is an IL-6R antagonist. In some instances, the IL-6R antagonist is tocilizumab. In some instances, tocilizumab is administered to the subject as a single dose of about 8 mg/kg (e.g., 8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, ±1 mg/kg, ±1.5 mg/kg, or ±2 mg/kg; e.g., 8 mg/kg), and wherein the single dose does not exceed 800 mg. In some instances, tocilizumab is administered to the subject as a single dose of about 12 mg/kg (e.g., 12 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, ±1 mg/kg, ±1.5 mg/kg, or ±2 mg/kg; e.g., 12 mg/kg), and wherein the single dose does not exceed 800 mg. In some instances, tocilizumab is administered intravenously.

### IV. Therapeutic Agents

### A. Mosunetuzumab

The invention provides mosunetuzumab, a bispecific antibody that binds to CD20 and CD3, useful for treating a CD20-positive cell proliferative disorder. In some instances, the CD20-positive cell proliferative disorder is a relapsed and/or refractory (R/R) non-Hodgkin's lymphoma (NHL) (e.g., R/R follicular lymphoma (FL), R/R transformed FL (trFL), or R/R diffuse large B cell lymphoma (DLBCL)).

Mosunetuzumab has an anti-CD20 arm having a first binding domain comprising the following six hypervariable regions (HVRs): (a) an HVR-H1 comprising the amino acid sequence of GYTFTSYNMH (SEQ ID NO: 1); (b) an HVR-H2 comprising the amino acid sequence of AIYPGNGDTSYNQKFKG (SEQ ID NO: 2); (c) an HVR-H3 comprising the amino acid sequence of VVYYSNSYWYFDV (SEQ ID NO: 3); (d) an HVR-L1 comprising the amino acid sequence of RASSSVSYMH (SEQ ID NO: 4); (e) an HVR-L2 comprising the amino acid sequence of APSNLAS (SEQ ID NO: 5); and (f) an HVR-L3 comprising the amino acid sequence of QQWSFNPPT (SEQ ID NO: 6). Mosunetuzumab comprises an anti-CD20 arm comprising a first binding domain comprising the heavy chain framework regions FR-H1, FR-H2, FR-H3, and FR-H4 comprising the sequences of SEQ ID NOs: 17-20, respectively, and the light chain framework regions FR-L1, FR-L2, FR-L3, and FR-L4 comprising the sequences of SEQ ID NOs: 21-24, respectively. Mosunetuzumab comprises an anti-CD20 arm comprising a first binding domain comprising (a) a heavy chain variable (VH) domain comprising the amino acid sequence of SEQ ID NO: 7; and (b) a light chain variable (VL) domain comprising the amino acid sequence o, SEQ ID NO: 8.

Mosunetuzumab has an anti-CD3 arm having a second binding domain comprising the following six HVRs: (a) an HVR-H1 comprising the amino acid sequence of NYYIH (SEQ ID NO: 9); (b) an HVR-H2 comprising the amino acid sequence of WIYPGDGNTKYNEKFKG (SEQ ID NO: 10); (c) an HVR-H3 comprising the amino acid sequence of DSYSNYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WASTRES (SEQ ID NO: 13); and (f) an HVR-L3 comprising the amino acid sequence of TQSFILRT (SEQ ID NO: 14). Mosunetuzumab comprises an anti-CD3 arm comprising a second binding domain comprising the heavy chain framework regions FR-H1, FR-H2, FR-H3, and FR-H4 comprising the sequences of SEQ ID NOs: 25-28, respectively, and the light chain framework regions FR-L1, FR-L2, FR-L3, and FR-L4 comprising the sequences of SEQ ID NOs: 29-32, respectively. Mosunetuzumab comprises an anti-CD3 arm comprising a second binding domain comprising (a) a VH domain comprising an amino acid sequence having the amino acid sequence of SEQ ID NO: 15; and (b) a VL domain comprising the amino acid sequence of SEQ ID NO: 16.

Mosunetuzumab has the International Nonproprietary Names for Pharmaceutical Substances (INN) List 117 (WHO Drug Information, Vol. 31, No. 2, 2017, p. 303), or CAS Registry No. 1905409-39-3, and having (1) an anti-CD20 arm comprising the heavy chain and light chain sequences of SEQ ID NOs: 33 and 34, respectively; and (2) an anti-CD3 arm comprising the heavy chain and light chain sequences of SEQ ID NOs: 35 and 36, respectively. Mosunetuzumab comprises (1) an anti-CD20 arm comprising a first binding domain comprising a heavy chain comprising the amino acid sequence of SEQ ID NO: 33 and a light chain comprising the amino acid sequence of SEQ ID NO: 34 and (2) an anti-CD3 arm comprising a second binding domain comprising a heavy chain comprising the amino acid sequence of SEQ ID NO: 35 and a light chain comprising the amino acid sequence of SEQ ID NO: 36.

Amino acid sequences of mosunetuzumab are summarized in Table 2 below.

TABLE 2
Sequence IDs for Mosunetuzumab

| CD3 Arm | | CD20 Arm | |
|---|---|---|---|
| SEQ ID NO: | Description | SEQ ID NO: | Description |
| 9 | CD3 HVR-H1 | 1 | CD20 HVR-H1 |
| 10 | CD3 HVR-H2 | 2 | CD20 HVR-H2 |
| 11 | CD3 HVR-H3 | 3 | CD20 HVR-H3 |
| 12 | CD3 HVR-L1 | 4 | CD20 HVR-L1 |
| 13 | CD3 HVR-L2 | 5 | CD20 HVR-L2 |
| 14 | CD3 HVR-L3 | 6 | CD20 HVR-L3 |
| 15 | CD3 VH | 7 | CD20 VH |
| 16 | CD3 VL | 8 | CD20 VL |
| 35 | CD3 heavy chain | 33 | CD20 heavy chain |
| 36 | CD3 light chain | 34 | CD20 light chain |

Mosunetuzumab may be produced using recombinant methods and compositions, for example, as described in U.S. Pat. No. 4,816,567.

### B. Additional Therapeutic Agents

In some instances, the methods described herein include administering mosunetuzumab in combination with one or more additional therapeutic agents. In some instances, the one or more additional therapeutic agents may reduce the rate or the severity of cytokine release syndrome (CRS). In some instances, the one or more additional therapeutic agents may prevent symptoms associated with CRS.

In particular instances, the additional therapeutic agent used to reduce the rate or severity of CRS or prevent symptoms associated with CRS is a corticosteroid (e.g., dexamethasone (CAS #: 50-02-2), prednisone (CAS #: 53-03-2), prednisolone (CAS #50-42-8), or methylprednisolone (CAS #: 83-43-2)) or an IL-6R antagonist (e.g., tocilizumab (CAS #: 375823-41-9), sarilumab (CAS #: 1189541-98-7), vobarilizumab (ALX-0061; CAS #: 1628814-88-9), satralizumab (SA-237; CAS #: 1535963-91-7), and variants thereof).

In some instances, the additional therapeutic agent is tocilizumab. In some instances, the additional therapeutic agent is a corticosteroid. In some instances, the corticosteroid is dexamethasone. In some instances, the corticosteroid is prednisone. In some instances, the corticosteroid is methylprednisolone.

In some instances, the one or more additional therapeutic agents is an anti-pyretic, e.g., acetaminophen or paracetamol. Acetaminophen or paracetamol has the CAS #: 103-90-2.

In some instances, the one or more additional therapeutic agents is an anti-histamine, e.g., diphenhydramine. Diphenhydramine has the CAS #: 58-73-1. In some embodiments, diphenhydramine is diphenhydramine hydrochloride (CAS #: 147-24-0).

In instances for which the methods described herein involve a combination therapy, such as a particular combination therapy noted above, the combination therapy encompasses the administration of mosunetuzumab with one or more additional therapeutic agents, and such co-administration may be combined administration (where two or more therapeutic agents are included in the same or separate formulations) or separate administration, in which case, the administration of mosunetuzumab can occur prior to, simultaneously, and/or following, administration of the additional therapeutic agent or agents.

In some embodiments, the one or more additional therapeutic agents administered in combination with mosunetuzumab is a corticosteroid, an anti-histamine, and/or an anti-pyretic. In some embodiments, the corticosteroid, anti-histamine, and/or anti-pyretic is administered to the subject as a premedication for mosunetuzumab administration.

In instances for which the methods described herein involve a combination therapy, such as a particular combination therapy noted above, the combination therapy encompasses the administration of mosunetuzumab with one or more additional therapeutic agents, and such co-administration may be combined administration (where two or more therapeutic agents are included in the same or separate formulations) or separate administration, in which case, the administration of mosunetuzumab can occur prior to, simultaneously, and/or following, administration of the additional therapeutic agent or agents.

In some embodiments, the one or more additional therapeutic agents administered in combination with mosunetuzumab is a corticosteroid, an anti-histamine, and/or an anti-pyretic. In some embodiments, the corticosteroid, anti-histamine, and/or anti-pyretic is administered to the subject as a premedication for mosunetuzumab administration.

### V. Pharmaceutical Compositions and Formulations

Mosunetuzumab described herein can be used in pharmaceutical compositions and formulations. Pharmaceutical compositions and formulations of mosunetuzumab and/or other therapeutic agents describe herein (e.g., dexamethasone, methylprednisolone, prednisone, acetaminophen, paracetamol, and diphenhydramine) can be prepared by mixing one, two, or more agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The formulation herein may also contain more than one active ingredient as necessary for the particular indication being treated, preferably those with complementary activities that do not adversely affect each other. For example, it may be desirable to further provide an additional therapeutic agent (e.g., a corticosteroid, an anti-histamine, an anti-pyretic, a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes.

In some embodiments, mosunetuzumab is formulated for administration intravenously (e.g., intravenous infusion). In some embodiments, dexamethasone is formulated for administration intravenously. In some embodiments, dexamethasone is formulated for administration intravenously or orally. In some embodiments, methylprednisolone is formulated for administration intravenously or orally. In some embodiments, acetaminophen or paracetamol is formulated for administration orally. In some embodiments, diphenhydramine is formulated for administration orally or intravenously.

### VI. Kits and Articles of Manufacture

In another aspect of the invention, a kit or an article of manufacture containing materials useful for the treatment, prevention, and/or diagnosis of the disorders described above is provided. The kit or article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be a vial having a stopper pierceable by a hypodermic injection needle). At least one active agent in the composition is mosunetuzumab, a corticosteroid, an anti-histamine, or an anti-pyretic described herein. The label or package insert indicates that the composition is used for treating the condition of choice (e.g., an R/R NHL, e.g., R/R FL, (e.g., a Grade 1, 2, 3a, but not 3b, R/R FL), R/R trFL, or R/R DLBCL) and further includes information related to at least one of the dosing regimens described herein. Moreover, the kit or article of manufacture may comprise (a) a first container with a composition contained therein, wherein the composition comprises an anti-CD20/anti-CD3 bispecific antibody described herein; and (b) a second container with a composition contained therein, wherein the composition comprises a further cytotoxic or otherwise therapeutic agent. Alternatively, or additionally, the kit or article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following are examples of the methods of the invention. It is understood that various other embodiments may be practiced, given the general description provided above.

### Example 1. Mosunetuzumab is Efficacious and Well Tolerated in Patients Aged <65 Years and ≥65 Years with Relapsed/Refractory (R/R) Follicular Lymphoma (FL) and ≥2 Prior Therapies: Subgroup Analysis of a Pivotal Phase II Study

This study compares the safety, tolerability, and pharmacokinetics of step-load-base dosing of mosunetuzumab in patients younger than 65 years and patients aged 65 years or older with relapsed or refractory (R/R) follicular lymphoma (FL). All patients had Grade (Gr) 1-3a R/R FL and Eastern Cooperative Oncology Group (ECOG) performance status (PS) 0-1, and were refractory to or had relapsed after ≥2 prior therapies, including an anti-CD20 antibody and an alkylating agent. Follicular lymphoma typically affects elderly patients, who often have a decline in immune function and are more vulnerable to treatment-related toxicities than younger patients.

### Method

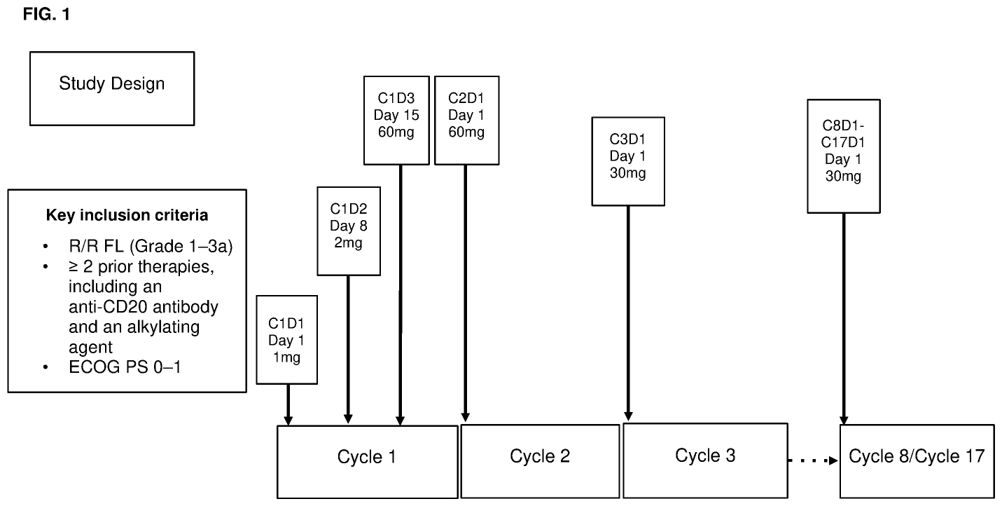

Patients were administered three escalating doses of 1 mg, 2 mg, and 60 mg of mosunetuzumab in a first dosing cycle, a single dose of 60 mg of mosunetuzumab in a second dosing cycle, and additional single doses of 30 mg of mosunetuzumab in additional dosing cycles thereafter. FIG. 1 depicts an overview of the dosing regimen of the study. In particular, intravenous (IV) mosunetuzumab was administered in 21-day dosing cycles with a first dosing cycle (Cycle 1 (C1)) having a first dose (C1D1 dose) of 1 mg administered on Day 1 of Cycle 1; a second dose (C1D2 dose) of 2 mg administered on Day 8 of Cycle 1; a third dose (C1D3 dose) of 60 mg administered on Day 15 of Cycle 1; a second dosing cycle (Cycle 2) having a first dose (C2D1 dose) of 60 mg administered on Day 1 of Cycle 2; and additional dosing cycles (Cycle 3+) each having a single dose (C3D1+) of 30 mg administered on Day 1 of each respective additional dosing cycle.

Corticosteroid premedication (IV dexamethasone 20 mg or IV methylprednisolone 80 mg) was administered one hour before each dose of mosunetuzumab in Cycles 1 and 2, and was optional from Cycle 3 onwards.

Patients with a complete response by dosing cycle 8 (C8) completed therapy; patients with a partial response or stable disease continued treatment for up to 17 dosing cycles (C17), unless disease progression or unacceptable toxicity occurred. The primary endpoint was complete response (as best response) rate (CR) by positron emission tomography (PET)/computed tomography (CT) assessed by Independent Review Committee using standard response criteria (Cheson et al. Journal of Clinical Oncology. 25:579-586, 2007). Secondary endpoints included objective response rate (ORR), duration of response (DoR), duration of CR (DoCR), progression-free survival (PFS), and safety and tolerability. Cytokine release syndrome (CRS) was graded using ASTCT criteria (Lee et al. Biology of Blood and Marrow Transplantation. 25(4): 625-638, 2019).

At data cut-off (Aug. 27, 2021), 90 patients had received mosunetuzumab. Of these, 60 (67%) were aged <65 years and 30 (33%) were ≥65 years (Table 3).

TABLE 3
Baseline Patient Characteristics

| | <65 years | | ≥65 years | |
|---|---|---|---|---|
| Characteristic at study entry | (N = 60) | | (N = 30) | |
| Median age, years (range) | 56 | (29-64) | 71 | (65-90) |
| Female, n (%) | 19 | (32) | 16 | (53) |
| ECOG PS, n (%) | | | | |
| 0 | 36 | (60) | 17 | (57) |
| 1 | 24 | (40) | 13 | (43) |
| FLIPI score, n (%) | | | | |
| 0-2 | 36 | (60) | 14 | (47) |
| 3-5 | 24 | (40) | 16 | (53) |
| Ann Arbor stage, n (%) | | | | |
| I/II | 12 | (20) | 9 | (30) |
| III/IV | 48 | (80) | 21 | (70) |
| Number of prior therapies, median (range) | 3 | (2-10) | 3 | (2-8) |
| Prior therapy, n (%) | | | | |
| Refractory to prior anti-CD20 | 48 | (80) | 23 | (77) |
| Double refractory to prior anti-CD20 and | 35 | (58) | 13 | (43) |
| alkylator therapy | | | | |
| POD24 | 36 | (60) | 11 | (37) |

ECOG PS = Eastern Cooperative Oncology Group performance status; FLIPI = Follicular Lymphoma International Prognostic Index; POD24 = progression of disease within 24 months

Median observation time was 18.4 months in patients aged ≤65 years, and 18.1 months in patients aged ≥65 years. Of the patients aged ≤65 years, 30 patients completed initial treatment and 30 patients discontinued treatment (21 due to progressive disease, three due to AEs, and three due to other reasons). Of the patients aged ≥65 years, 24 patients completed initial treatment and six patients discontinued treatment (four due to progressive disease, one due to an AE, and one due to other reasons). Median duration of treatment was 4.9 months for patients aged ≤65 years and 5.1 months for patients ≥65 years. See Table 4 for details.

TABLE 4
Mosunetuzumab Exposure Summary

| | <65 Years | | ≥65 Years | |
|---|---|---|---|---|
| Exposure | (N = 60) | | (N = 30) | |
| Median number of cycles, n (range) | 8 | (1-17) | 8 | (2-17) |
| Number of cycles, n (%) | | | | |
| <8 | 18 | (30) | 3 | (10) |
| 8 | 32 | (53) | 21 | (70) |
| 9-16 | 4 | (7) | 1 | (3) |
| 17 | 6 | (10) | 5 | (17) |
| Median dose intensity, % | 99 | | 98 | |
| Patients with >90% dose intensity, n (%) | 49 | (82) | 24 | (80) |

| | | |
|---|---|---|
| Median treatment duration, months | 4.9 | 5.1 |

### Efficacy Results

High response rates with associated durability were achieved in both age subgroups. Complete response rate and objective response rate were high across the study and were numerically higher in patients aged ≥65 years than in those aged <65 years. (Table 5).

TABLE 5
Efficacy Outcomes

| | <65 years | | ≥65 years | |
|---|---|---|---|---|
| Efficacy endpoint | (N = 60) | | (N = 30) | |
| CR, % (95% CI) | 55.0 | (41.6-67.9) | 70.0 | (50.6-85.3) |
| ORR, % (95% CI) | 76.7 | (64.0-86.6) | 86.7 | (69.3-96.2) |
| DoR | | | | |
| Median, months (95% CI) | 22.8 | (8.7-NE) | 18.7 | (9.4-NE) |
| 18-month event-free rate, | 58.5 | (43.3-73.8) | 53.5 | (30.8-76.3) |
| % (95% CI) | | | | |
| DoCR | | | | |
| Median, months (95% CI) | NE | (9.1-NE) | 18.7 | (13.7-NE) |
| 18-month event-free rate, | 74.0 | (58.5-89.6) | 63.6 | (38.7-88.4) |
| % (95% CI) | | | | |
| PFS | | | | |
| Median, months (95% CI) | 12.0 | (8.4-NE) | 17.9 | (12.0-NE) |
| 18-month event-free rate, | 48.1 | (34.1-62.0) | 45.4 | (22.4-68.4) |
| % (95% CI) | | | | |

CI = confidence interval; CR = complete response; DoR = duration of response; DoCR = duration of complete response; NE = not evaluable; ORR = objective response rate; PFS = progression-free survival.

High response rates and durable responses were achieved in both age subgroups, with a CR rate of 70% observed in patients aged ≥65 years. The median PFS for patients aged ≥65 years was 17.9 months (95% CI: 12.0-NE), highly consistent with the PFS reported for the overall study population. Median PFS in patients aged <65 years was 12.0 months (95% CI: 8.4-NE).

### Pharmacokinetics and Biomarker Results

The pharmacokinetic disposition of mosunetuzumab was comparable across the age groups. Age was tested as a continuous variable in a population pharmacokinetic (PK) model and was not found to be significantly associated with mosunetuzumab PK parameters (p>0.05). Exposure (AUC [area under the curve] 0-42 days) was similar between patients aged <65 compared to patients aged ≥65 years.

Levels of CD20 (based on dual CD20+ PAX5+ immunohistochemistry [IHC]), intratumoral T-cells (CD8+ IHC), and peripheral T cells, B cells and natural killer cells (by flow cytometry) were similar at baseline between patients aged <65 compared to patients aged ≥65 years.

### Safety Results

The rates of all-Grade and Grade 3/4 adverse events (AEs) were comparable between age groups. However, the rate of serious AEs (SAEs) was lower in patients aged 65 years compared with those aged <65 years (37% versus 52%; Table 6).

TABLE 6
Safety Overview

| | <65 Years | ≥65 Years |
|---|---|---|
| Event, n (%) | (N = 60) | (N = 30) |
| Any AE | 60 (100) | 30 (100) |
| Related to mosunetuzumab | 56 (93) | 27 (90) |
| Grade 3-4 AE | 41 (68) | 22 (73) |
| Related to mosunetuzumab | 30 (50) | 16 (53) |
| Grade 5 (fatal) AE* | 0 (0) | 1 (3)^{†} |
| Related to mosunetuzumab | 0 (0) | 0 (0) |
| Leading to discontinuation | 3 (5) | 1 (3) |
| Related to mosunetuzumab | 1 (2) | 1 (3) |
| Any SAE^{‡} | 31 (52) | 11 (37) |
| Related to mosunetuzumab | 21 (35) | 9 (30) |

*Excluding Grade 5 malignant neoplasm progression;
^{†}Unexplained death (occurred 31 days after treatment discontinuation and without antecedent signs or symptoms);
^{‡}Excluding Grade 5 PD
AE = adverse event; PD = progressive disease; SAE = serious adverse event

Two mosunetuzumab-related neurological AEs (NAEs) potentially consistent with immune effector cell-associated neurotoxicity syndrome (ICANS) were noted in each age subgroup (all NAEs were Grade 1/2): in patients age <65 years of age: confusional state (n=1; Grade 1), disturbance in attention (n=1; Grade 1); in patients 65 years of age: confusional state and cognitive disorder (n=1; Grade 1), confusional state (n=1; Grade 2).

There were no cases of aphasia, seizures, encephalopathy, or cerebral edema.

Rates of neutropenia and SAEs of infection, which were adverse events of special interest in the study, were similar between age subgroups (Table 7)

Thirty-four events of neutropenia were reported in patients aged <65 years, of which 97% were resolved; 20 events were reported in patients aged 65 years, all events were resolved.

TABLE 7
Adverse Events of Special Interest

| | <65 Years | ≥65 Years |
|---|---|---|
| Event, n (%) | (N = 60) | (N = 30) |
| Infection SAE | 13 (22) | 5 (17) |
| Grade 3 or 4 | 10 (17) | 3 (10) |
| Neutropenia | 19 (32) | 7 (23) |
| Grade 3 or 4 | 17 (28) | 7 (23) |

SAE = serious adverse event

CRS events were mostly low grade (<Grade 3 according to American Society for Transplantation and Cellular Therapy (ASTCT) criteria (Lee et al. Biology of Blood and Marrow Transplantation. 25(4): 625-638, 201)) and all were resolved. CRS events were numerically less frequent in patients aged 65 years in comparison with those <65 years (30% versus 52%; Table 8).

TABLE 8
Cytokine Release Syndrome (CRS) Overview

| | <65 Years | | ≥65 Years | |
|---|---|---|---|---|
| CRS event | (N = 60) | | (N = 30) | |
| Patients with a CRS event, n (%) | 31 | (52) | 9 | (30) |
| CRS Grade, n (%) | | | | |
| Grade 1 | 17 | (28) | 6 | (20) |
| Grade 2 | 13 | (22) | 2 | (7) |
| Grade 3 | 0 | | 1 | (3) |
| Grade 4 | 1 | (2) | 0 | |
| Median duration of CRS events, days | 3 | (1-29) | 3 | (1-8) |
| (range) | | | | |
| Management approach, number of | | | | |
| patients treated/number of patients | | | | |
| with CRS of any Grade (%) | | | | |
| Tocilizumab | 5/31 | (16) | 2/9 | (22) |
| Corticosteroids | 7/31 | (23) | 3/9 | (33) |

Low rates of discontinuations due to adverse events (AEs) were observed. In the 65 years subgroup, 3% of patients discontinued treatment due to AEs and no treatment-related fatal events were reported

### EMBODIMENTS

Some embodiments of the technology described herein can be defined according to any of the following numbered embodiments:
1. A method of treating a subject aged about 65 years or older having a relapsed or refractory (R/R) non-Hodgkin's lymphoma (NHL) comprising intravenously administering to the subject mosunetuzumab in a dosing regimen comprising at least a first dosing cycle, a second dosing cycle, and a third dosing cycle, wherein:
   (a) the first three-dose dosing cycle comprises a first dose (C1D1), a second dose (C1D2), and a third dose (C1D3) of mosunetuzumab, wherein the C1 D1 is about 1 mg, the C1 D2 is about 2 mg, and the C1 D3 is about 60 mg;
   (b) the second dosing cycle comprises a single dose (C2D1) of mosunetuzumab, wherein the C2D1 is about 60 mg; and
   (c) the third dosing cycle comprises a single dose (C3D1) of mosunetuzumab, wherein the C3D1 is about 30 mg.
2. Mosunetuzumab for use in treating a subject aged about 65 years or older having a relapsed or refractory (R/R) non-Hodgkin's lymphoma (NHL), wherein mosunetuzumab is to be administered intravenously to the subject in a dosing regimen comprising at least a first dosing cycle, a second dosing cycle, and a third dosing cycle, wherein:
   (a) the first three-dose dosing cycle comprises a first dose (C1D1), a second dose (C1D2), and a third dose (C1D3) of mosunetuzumab, wherein the C1 D1 is about 1 mg, the C1 D2 is about 2 mg, and the C1 D3 is about 60 mg;
   (b) the second dosing cycle comprises a single dose (C2D1) of mosunetuzumab, wherein the C2D1 is about 60 mg; and
   (c) the third dosing cycle comprises a single dose (C3D1) of mosunetuzumab, wherein the C3D1 is about 30 mg.
3. Use of mosunetuzumab for treating a subject aged about 65 years or older having a relapsed or refractory (R/R) non-Hodgkin's lymphoma (NHL), wherein mosunetuzumab is to be administered intravenously to the subject in a dosing regimen comprising at least a first dosing cycle, a second dosing cycle, and a third dosing cycle, wherein:
   (a) the first three-dose dosing cycle comprises a first dose (C1D1), a second dose (C1D2), and a third dose (C1D3) of mosunetuzumab, wherein the C1 D1 is about 1 mg, the C1 D2 is about 2 mg, and the C1 D3 is about 60 mg;
   (b) the second dosing cycle comprises a single dose (C2D1) of mosunetuzumab, wherein the C2D1 is about 60 mg; and
   (c) the third dosing cycle comprises a single dose (C3D1) of mosunetuzumab, wherein the C3D1 is about 30 mg.
4. Use of mosunetuzumab in the manufacture of a medicament for use in treating a subject aged about 65 years or older having a relapsed or refractory (R/R) non-Hodgkin's lymphoma (NHL), wherein mosunetuzumab is to be administered intravenously to the subject in a dosing regimen comprising at least a first dosing cycle, a second dosing cycle, and a third dosing cycle, wherein:
   (a) the first three-dose dosing cycle comprises a first dose (C1D1), a second dose (C1D2), and a third dose (C1D3) of mosunetuzumab, wherein the C1 D1 is about 1 mg, the C1 D2 is about 2 mg, and the C1 D3 is about 60 mg;
   (b) the second dosing cycle comprises a single dose (C2D1) of mosunetuzumab, wherein the C2D1 is about 60 mg; and
   (c) the third dosing cycle comprises a single dose (C3D1) of mosunetuzumab, wherein the C3D1 is about 30 mg.
5. The method, mosunetuzumab for use, or use of any one of embodiments 1-4, wherein the subject has relapsed after, or is refractory to, two or more prior lines of therapy.
6. The method, mosunetuzumab for use, or use of embodiment 5, wherein the two or more prior lines of therapy comprise an anti-CD20 monoclonal antibody, an alkylating agent, or both an anti-CD20 monoclonal antibody and an alkylating agent.
7. The method, mosunetuzumab for use, or use of any one of embodiments 1-6, wherein the R/R NHL is R/R follicular lymphoma (FL), R/R transformed FL (trFL), or R/R diffuse large B cell lymphoma (DLBCL).
8. The method, mosunetuzumab for use, or use of embodiment 7, wherein the R/R NHL is R/R FL.
9. The method, mosunetuzumab for use, or use of embodiment 8, wherein the R/R FL is histologically documented as Grade 1, 2, or 3a, but not 3b according to the World Health Organization classification of lymphoid neoplasms (as referenced in Swerdlow S H, et al. Blood 2016; 127:2375-90).
10. The method, mosunetuzumab for use, or use of any one of embodiments 1-9, wherein the first, second, and third dosing cycles are 21-day dosing cycles.
11. The method of any one of embodiments 1 and 5-10, wherein the method comprises administering the C1 D1, the C1 D2, and the C1 D3 on Days 1, 8, and 15, respectively, of the first dosing cycle.
12. The method of any one of embodiments 1 and 5-11, wherein the method comprises administering the C2D1 on Day 1 of the second dosing cycle.
13. The method of any one of embodiments 1 and 5-12, wherein the method comprises administering the C3D1 on Day 1 of the third dosing cycle.
14. The mosunetuzumab for use or use of any one of embodiments 2-10, wherein the C1 D1, the C1 D2, and the C1 D3 are to be administered to the subject on Days 1, 8, and 15, respectively, of the first dosing cycle.
15. The mosunetuzumab for use or use of any one of embodiments 2-10 and 14, wherein the C2D1 is to be administered to the subject on Day 1 of the second dosing cycle.
16. The mosunetuzumab for use or use of any one of embodiments 2-10, 14, and 15, wherein the C3D1 is to be administered to the subject on Day 1 of the third dosing cycle.
17. The method, mosunetuzumab for use, or use of any one of embodiments 1-16, wherein the dosing regimen further comprises one or more additional dosing cycles.
18. The method, mosunetuzumab for use, or use of embodiment 17, wherein the dosing regimen comprises five to 14 additional dosing cycles.
19. The method, mosunetuzumab for use, or use of embodiment 18, wherein the dosing regimen comprises five additional dosing cycles.
20. The method, mosunetuzumab for use, or use of embodiment 19, wherein the dosing regimen comprises 14 additional dosing cycles.
21. The method, mosunetuzumab for use, or use of any one of embodiments 17-20, wherein the one or more additional dosing cycles are each 21-day dosing cycles.
22. The method, mosunetuzumab for use, or use of any one of embodiments 17-21, wherein each of the one or more of the additional dosing cycles comprises an additional single dose of mosunetuzumab.
23. The method, mosunetuzumab for use, or use of embodiment 22, wherein the additional single dose of mosunetuzumab is administered or is to be administered to the subject on Day 1 of each respective additional dosing cycle.
24. The method, mosunetuzumab for use, or use of embodiment 22 or 23, wherein the additional single dose of mosunetuzumab is about 30 mg.
25. The method, mosunetuzumab for use, or use of any one of embodiments 1-24, wherein mosunetuzumab is administered or is to be administered by intravenous infusion.
26. A method of treating a population of subjects aged about 65 years or older having an R/R NHL comprising intravenously administering to each subject mosunetuzumab in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third dosing cycle comprises a single dose (C3D1) of mosunetuzumab administered on Day 1 of the third dosing cycle, wherein the C3D1 is about 30 mg.
27. Mosunetuzumab for use in treating a population of subjects aged about 65 years or older having an R/R NHL, wherein mosunetuzumab is to be administered intravenously to each subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg.
28. Use of mosunetuzumab for treating a population of subjects aged about 65 years or older having an R/R NHL, wherein mosunetuzumab is to be administered intravenously to each subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg.
29. Use of mosunetuzumab in the manufacture of a medicament for use in treating a population of subjects aged about 65 years or older having an R/R NHL, wherein mosunetuzumab is to be administered intravenously to each subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg.
30. A method of treating a population of subjects aged about 65 years or older having an R/R NHL comprising intravenously administering to each subject mosunetuzumab in a dosing regimen comprising eight 21-day dosing cycles, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third to eighth dosing cycles each comprises a single dose (C3D1-C8D1) of mosunetuzumab administered on Day 1 of each respective dosing cycle, wherein each of the C3D1-C8D1 is about 30 mg.
31. Mosunetuzumab for use in treating a population of subjects aged about 65 years or older having an R/R NHL, wherein mosunetuzumab is to be administered intravenously to each subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg.
32. Use of mosunetuzumab for treating a population of subjects aged about 65 years or older having an R/R NHL, wherein mosunetuzumab is to be administered intravenously to each subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg.
33. Use of mosunetuzumab in the manufacture of a medicament for use in treating a population of subjects aged about 65 years or older having an R/R NHL, wherein mosunetuzumab is to be administered intravenously to each subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg.
34. A method of treating a population of subjects aged about 65 years or older having an R/R NHL comprising intravenously administering to each subject mosunetuzumab in a dosing regimen comprising 17 21-day dosing cycles, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third to 17^{th} dosing cycles each comprises a single dose (C3D1-C17D1) of mosunetuzumab administered on Day 1 of each respective dosing cycle, wherein each of the C3D1-C17D1 is about 30 mg.
35. Mosunetuzumab for use in treating a population of subjects aged about 65 years or older having an R/R NHL, wherein mosunetuzumab is to be administered intravenously to each subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg.
36. Use of mosunetuzumab for treating a population of subjects aged about 65 years or older having an R/R NHL, wherein mosunetuzumab is to be administered intravenously to each subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg.
37. Use of mosunetuzumab in the manufacture of a medicament for use in treating a population of subjects aged about 65 years or older having an R/R NHL, wherein mosunetuzumab is to be administered intravenously to each subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg.
38. The method, mosunetuzumab for use, or use of any one of embodiments 26-37, wherein each subject in the population of subjects has relapsed after, or is refractory to, two or more prior lines of therapy.
39. The method, mosunetuzumab for use, or use of embodiment 38, wherein the two or more prior lines of therapy comprise an anti-CD20 monoclonal antibody, an alkylating agent, or both an anti-CD20 monoclonal antibody and an alkylating agent.
40. The method, mosunetuzumab for use, or use of any one of embodiments 26-39, wherein the R/R NHL is R/R FL, R/R trFL, or R/R DLBCL.
41. The method, mosunetuzumab for use, or use of embodiment 40, wherein the R/R NHL is R/R FL.
42. The method, mosunetuzumab for use, or use of embodiment 41, wherein the R/R FL is histologically documented as Grade 1, 2, or 3a, but not 3b according to the World Health Organization classification of lymphoid neoplasms (as referenced in Swerdlow S H, et al. Blood 2016; 127:2375-90).
43. The method, mosunetuzumab for use, or use of any one of embodiments 26-42, wherein mosunetuzumab is administered or is to be administered by intravenous infusion.
44. The method, mosunetuzumab for use, or use of any one of embodiments 26-43, wherein the objective response rate in the population of subjects is 65%.
45. The method, mosunetuzumab for use, or use of embodiment 44, wherein the objective response rate in the population of subjects is 75%.
46. The method, mosunetuzumab for use, or use of embodiment 45, wherein the objective response rate in the population of subjects is 85%.
47. The method, mosunetuzumab for use, or use of any one of embodiments 26-43, wherein the complete response rate in the population of subjects is 50%.
48. The method, mosunetuzumab for use, or use of embodiment 47, wherein the complete response rate in the population of subjects is 60%.
49. The method, mosunetuzumab for use, or use of embodiment 48, wherein the complete response rate in the population of subjects is 70%.
50. The method, mosunetuzumab for use, or use of any one of embodiments 26-43, wherein 40% of subjects having an objective response maintained remission for 18 months.
51. The method, mosunetuzumab for use, or use of embodiment 50, wherein 50% of subjects having an objective response maintained remission for 18 months.
52. The method, mosunetuzumab for use, or use of any one of embodiments 26-43, wherein 50% of subjects having a complete response maintained complete remission for 18 months.
53. The method, mosunetuzumab for use, or use of embodiment 52, wherein 60% of subjects having a complete response maintained complete remission for 18 months.
54. The method, mosunetuzumab for use, or use of any one of embodiments 26-43, wherein the median duration of response in the population of subjects is 9 months.
55. The method, mosunetuzumab for use, or use of embodiment 54, wherein the median duration of response in the population of subjects is 15 months.
56. The method, mosunetuzumab for use, or use of any one of embodiments 26-43, wherein the median duration of complete response in the population of subjects is 12 months.
57. The method of embodiment 56, wherein the median duration of complete response in the population of subjects is 16 months.
58. The method, mosunetuzumab for use, or use of any one of embodiments 26-43, wherein 35% of subjects have a progression-free survival of 18 months.
59. The method, mosunetuzumab for use, or use of embodiment 58, wherein 45% of subjects have a progression-free survival of 18 months.
60. The method, mosunetuzumab for use, or use of any one of embodiments 26-43, wherein the median progression-free survival in the population of subjects is 12 months.
61. The method, mosunetuzumab for use, or use of embodiment 60, wherein the median progression-free survival in the population of subjects is 17 months.
62. The method, mosunetuzumab for use, or use of any one of embodiments 26-43, wherein the rate of serious adverse events, excluding Grade 5 malignant neoplasm progression, in the population of subjects is ≤45%.
63. The method, mosunetuzumab for use, or use of embodiment 62, wherein the rate of serious adverse events, excluding Grade 5 malignant neoplasm progression, in the population of subjects is ≤40%.
64. The method, mosunetuzumab for use, or use of any one of embodiments 26-43, wherein the rate of serious adverse events related to mosunetuzumab in the population of subjects is ≤40%.
65. The method, mosunetuzumab for use, or use of embodiment 64, wherein the rate of serious adverse events related to mosunetuzumab in the population of subjects is ≤30%.
66. The method, mosunetuzumab for use, or use of any one of embodiments 26-43, wherein the rate of cytokine release syndrome having a grade of 3 or higher (as defined by the American Society for Transplantation and Cellular Therapy, 2018; ASTCT) in the population of subjects is ≤5%.
67. The method, mosunetuzumab for use, or use of embodiment 66, wherein the rate of cytokine release syndrome having a grade of 3 or higher (as defined by the ASTCT) in the population of subjects is ≤3%.
68. The method, mosunetuzumab for use, or use of any one of embodiments 26-43, wherein the rate of cytokine release syndrome of any grade (as defined by the ASTCT) in the population of subjects is ≤40%.
69. The method, mosunetuzumab for use, or use of embodiment 68, wherein the rate of cytokine release syndrome of any grade (as defined by the ASTCT) in the population of subjects is ≤30%.
70. A method of achieving an objective response, a complete response, or progression-free survival in a subject aged about 65 years or older having an R/R NHL, wherein the method comprises intravenously administering to the subject mosunetuzumab in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third dosing cycle comprises a single dose (C3D1) of mosunetuzumab administered on Day 1 of the third dosing cycle, wherein the C3D1 is about 30 mg.
71. Mosunetuzumab for use in achieving an objective response, a complete response, or progression-free survival in a subject aged about 65 years or older having an R/R NHL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg.
72. Use of mosunetuzumab for achieving an objective response, a complete response, or progression-free survival in a subject aged about 65 years or older having an R/R NHL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg.
73. Use of mosunetuzumab in the manufacture of a medicament for use in achieving an objective response, a complete response, or progression-free survival in a subject aged about 65 years or older having an R/R NHL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg.
74. A method of achieving an objective response, a complete response, or progression-free survival in a subject aged about 65 years or older having an R/R NHL, wherein the method comprises intravenously administering to the subject mosunetuzumab in a dosing regimen comprising eight 21-day dosing cycles, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third to eighth dosing cycles each comprises a single dose (C3D1-C8D1) of mosunetuzumab administered on Day 1 of each respective dosing cycle, wherein each of the C3D1-C8D1 is about 30 mg.
75. Mosunetuzumab for use in achieving an objective response, a complete response, or progression-free survival in a subject aged about 65 years or older having an R/R NHL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg.
76. Use of mosunetuzumab for achieving an objective response, a complete response, or progression-free survival in a subject aged about 65 years or older having an R/R NHL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg.
77. Use of mosunetuzumab in the manufacture of a medicament for use in achieving an objective response, a complete response, or progression-free survival in a subject aged about 65 years or older having an R/R NHL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg.
78. A method of achieving an objective response, a complete response, or progression-free survival in a subject aged about 65 years or older having an R/R NHL, wherein the method comprises intravenously administering to the subject mosunetuzumab in a dosing regimen comprising 17 21-day dosing cycles, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third to 17^{th} dosing cycles each comprises a single dose (C3D1-C17D1) of mosunetuzumab administered on Day 1 of each respective dosing cycle, wherein each of the C3D1-C17D1 is about 30 mg.
79. Mosunetuzumab for use in achieving an objective response, a complete response, or progression-free survival in a subject aged about 65 years or older having an R/R NHL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg.
80. Use of mosunetuzumab for achieving an objective response, a complete response, or progression-free survival in a subject aged about 65 years or older having an R/R NHL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg.
81. Use of mosunetuzumab in the manufacture of a medicament for use in achieving an objective response, a complete response, or progression-free survival in a subject aged about 65 years or older having an R/R NHL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg.
82. The method, mosunetuzumab for use, or use of any one of embodiments 70-81, wherein the subject has relapsed after, or is refractory to, two or more prior lines of therapy.
83. The method, mosunetuzumab for use, or use of embodiment 82, wherein the two or more prior lines of therapy comprise an anti-CD20 monoclonal antibody, an alkylating agent, or both an anti-CD20 monoclonal antibody and an alkylating agent.
84. The method, mosunetuzumab for use, or use of any one of embodiments 70-83, wherein the R/R NHL is R/R FL, R/R trFL, or R/R DLBCL.
85. The method, mosunetuzumab for use, or use of embodiment 84, wherein the R/R NHL is R/R FL.
86. The method, mosunetuzumab for use, or use of embodiment 85, wherein the R/R FL is histologically documented as Grade 1, 2, or 3a, but not 3b according to the World Health Organization classification of lymphoid neoplasms (as referenced in Swerdlow S H, et al. Blood 2016; 127:2375-90).
87. The method, mosunetuzumab for use, or use of any one of embodiments 70-86, wherein mosunetuzumab is administered or is to be administered by intravenous infusion.
88. The method, mosunetuzumab for use, or use of any one of embodiments 70-87, wherein the subject achieves an objective response.
89. The method, mosunetuzumab for use, or use of any one of embodiments 70-87, wherein the subject achieves a complete response.
90. The method, mosunetuzumab for use, or use of any one of embodiments 70-87, wherein the progression-free survival is maintained for 12 months.
91. The method, mosunetuzumab for use, or use of embodiment 90, wherein the progression-free survival is maintained for 18 months.
92. The method, mosunetuzumab for use, or use of any one of embodiments 1-91, wherein
   (a) the method further comprises administering to the subject or to each subject in the population of subjects one or more additional therapeutic agents;
   (b) mosunetuzumab is to be administered to the subject or to each subject in the population of subjects in combination with one or more additional therapeutic agents; or
   (c) the medicament is formulated for administration to the subject or to each subject in the population of subjects in combination with one or more additional therapeutic agents.
93. The method, mosunetuzumab for use, or use of embodiment 92, wherein the one or more additional therapeutic agents comprises tocilizumab.
94. The method, mosunetuzumab for use, or use of embodiment 92 or 93, wherein the one or more additional therapeutic agents comprises a corticosteroid.
95. The method, mosunetuzumab for use, or use of embodiment 94, wherein the corticosteroid comprises dexamethasone or methylprednisolone.
96. The method, mosunetuzumab for use, or use of embodiment 94 or 95, wherein the corticosteroid is administered or is to be administered to the subject or to each subject in the population of subjects at least one hour prior to the administration of any dose of mosunetuzumab.
97. The method, mosunetuzumab for use, or use of any one of embodiments 94-96, wherein the corticosteroid is only administered or is only to be administered during dosing cycle 1 or dosing cycle 2.
98. The method, mosunetuzumab for use, or use of any one of embodiments 94-97, wherein the corticosteroid is administered or is to be administered intravenously.
99. The method, mosunetuzumab for use, or use of any one of embodiments 94-98, wherein the corticosteroid comprises dexamethasone and is administered or is to be administered at a dose of about 20 mg.
100. The method, mosunetuzumab for use, or use of any one of embodiments 94-98, wherein the corticosteroid comprises methylprednisolone and is administered or is to be administered at a dose of about 80 mg.
101. The method, mosunetuzumab for use, or use of any one of embodiments 92-100, wherein the one or more additional therapeutic agents comprises an anti-histamine.
102. The method, mosunetuzumab for use, or use of embodiment 101, wherein the anti-histamine is administered or is to be administered to the subject or to each subject in the population of subjects at least 30 minutes prior to the administration of any dose of mosunetuzumab.
103. The method, mosunetuzumab for use, or use of embodiment 101 or 102, wherein the anti-histamine is only administered or is only to be administered during dosing cycle 1 or dosing cycle 2.
104. The method, mosunetuzumab for use, or use of any one of embodiments 101-103, wherein the anti-histamine is administered or is to be administered orally or intravenously.
105. The method, mosunetuzumab for use, or use of any one of embodiments 101-104, wherein the anti-histamine comprises diphenhydramine hydrochloride and is administered or is to be administered at a dose of about 50-100 mg.
106. The method, mosunetuzumab for use, or use of any one of embodiments 92-105, wherein the one or more additional therapeutic agents comprises an anti-pyretic.
107. The method, mosunetuzumab for use, or use of embodiment 106, wherein the anti-pyretic is administered or is to be administered to the subject or to each subject in the population of subjects at least 30 minutes prior to the administration of any dose of mosunetuzumab.
108. The method, mosunetuzumab for use, or use of embodiment 106 or 107, wherein the anti-pyretic is only administered or is only to be administered during dosing cycle 1 or dosing cycle 2.
109. The method, mosunetuzumab for use, or use of any one of embodiments 106-108, wherein the anti-pyretic is administered or is to be administered orally.
110. The method, mosunetuzumab for use, or use of any one of embodiments 106-109, wherein the anti-pyretic comprises acetaminophen and is administered at a dose of about 500-1000 mg.
111. A method of treating a subject aged about 65 years or older having an R/R NHL comprising intravenously administering to the subject mosunetuzumab in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third dosing cycle comprises a single dose (C3D1) of mosunetuzumab administered on Day 1 of the third dosing cycle, wherein the C3D1 is about 30 mg,
wherein the subject has relapsed after, or is refractory to, two or more prior lines of therapy.
112. Mosunetuzumab for use in treating a subject aged about 65 years or older having an R/R NHL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg,
wherein the subject has relapsed after, or is refractory to, two or more prior lines of therapy.
113. Use of mosunetuzumab for treating a subject aged about 65 years or older having an R/R NHL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg,
wherein the subject has relapsed after, or is refractory to, two or more prior lines of therapy.
114. Use of mosunetuzumab in the manufacture of a medicament for use in treating a subject aged about 65 years or older having an R/R NHL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg,
wherein the subject has relapsed after, or is refractory to, two or more prior lines of therapy.
115. A method of treating a subject aged about 65 years or older having an R/R NHL comprising intravenously administering to the subject mosunetuzumab in a dosing regimen comprising eight 21-day dosing cycles, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third to eighth dosing cycles each comprises a single dose (C3D1-C8D1) of mosunetuzumab administered on Day 1 of each respective dosing cycle, wherein each of the C3D1-C8D1 is about 30 mg,
wherein the subject has relapsed after, or is refractory to, two or more prior lines of therapy.
116. Mosunetuzumab for use in treating a subject aged about 65 years or older having an R/R NHL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg,
wherein the subject has relapsed after, or is refractory to, two or more prior lines of therapy.
117. Use of mosunetuzumab for treating a subject aged about 65 years or older having an R/R NHL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg,
wherein the subject has relapsed after, or is refractory to, two or more prior lines of therapy.
118. Use of mosunetuzumab in the manufacture of a medicament for use in treating a subject aged about 65 years or older having an R/R NHL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg,
wherein the subject has relapsed after, or is refractory to, two or more prior lines of therapy.
119. A method of treating a subject aged about 65 years or older having an R/R NHL comprising intravenously administering to the subject mosunetuzumab in a dosing regimen comprising 17 21-day dosing cycles, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third to 17^{th} dosing cycles each comprises a single dose (C3D1-C17D1) of mosunetuzumab administered on Day 1 of each respective dosing cycle, wherein each of the C3D1-C17D1 is about 30 mg,
wherein the subject has relapsed after, or is refractory to, two or more prior lines of therapy.
120. Mosunetuzumab for use in treating a subject aged about 65 years or older having an R/R NHL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg,
wherein the subject has relapsed after, or is refractory to, two or more prior lines of therapy.
121. Use of mosunetuzumab for treating a subject aged about 65 years or older having an R/R NHL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg,
wherein the subject has relapsed after, or is refractory to, two or more prior lines of therapy.
122. Use of mosunetuzumab in the manufacture of a medicament for use in treating a subject aged about 65 years or older having an R/R NHL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg,
wherein the subject has relapsed after, or is refractory to, two or more prior lines of therapy.
123. A method of treating a subject aged about 65 years or older having an R/R NHL comprising intravenously administering to the subject mosunetuzumab in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third dosing cycle comprises a single dose (C3D1) of mosunetuzumab administered on Day 1 of the third dosing cycle, wherein the C3D1 is about 30 mg,
wherein the subject has relapsed after, or is refractory to, two or more prior lines of therapy, and
wherein the dosing regimen further comprises:
   (i) administering a corticosteroid, wherein a single dose of the corticosteroid is administered to the subject at least one hour prior to the administration of each of the C1 D1, C1 D2, C1 D3, and C2D1 of mosunetuzumab;
   (ii) administering an anti-histamine, wherein a single dose of the anti-histamine is administered to the subject at least 30 minutes prior to the administration of each of the C1 D1, C1 D2, C1 D3, and C2D1 of mosunetuzumab; and/or
   (iii) administering an anti-pyretic, wherein a single dose of the anti-pyretic is administered to the subject at least 30 minutes prior to the administration of each of the C1 D1, C1 D2, C1 D3, and C2D1 of mosunetuzumab.
124. Mosunetuzumab for use in treating a subject aged about 65 years or older having an R/R NHL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
125. Use of mosunetuzumab for treating a subject aged about 65 years or older having an R/R NHL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
126. Use of mosunetuzumab in the manufacture of a medicament for use in treating a subject aged about 65 years or older having an R/R NHL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
127. A method of treating a subject aged about 65 years or older having an R/R NHL comprising intravenously administering to the subject mosunetuzumab in a dosing regimen comprising eight 21-day dosing cycles, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third to eighth dosing cycles each comprises a single dose (C3D1-C8D1) of mosunetuzumab administered on Day 1 of each respective dosing cycle, wherein each of the C3D1-C8D1 is about 30 mg,
wherein the subject has relapsed after, or is refractory to, two or more prior lines of therapy, and
wherein the dosing regimen further comprises:
   (i) administering a corticosteroid, wherein a single dose of the corticosteroid is administered to the subject at least one hour prior to the administration of each of the C1 D1, C1 D2, C1 D3, and C2D1 of mosunetuzumab;
   (ii) administering an anti-histamine, wherein a single dose of the anti-histamine is administered to the subject at least 30 minutes prior to the administration of each of the C1 D1, C1 D2, C1 D3, and C2D1 of mosunetuzumab; and/or
   (iii) administering an anti-pyretic, wherein a single dose of the anti-pyretic is administered to the subject at least 30 minutes prior to the administration of each of the C1 D1, C1 D2, C1 D3, and C2D1 of mosunetuzumab.
128. Mosunetuzumab for use in treating a subject aged about 65 years or older having an R/R NHL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
129. Use of mosunetuzumab for treating a subject aged about 65 years or older having an R/R NHL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
130. Use of mosunetuzumab in the manufacture of a medicament for use in treating a subject aged about 65 years or older having an R/R NHL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
131. A method of treating a subject aged about 65 years or older having an R/R NHL comprising intravenously administering to the subject mosunetuzumab in a dosing regimen comprising 17 21-day dosing cycles, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third to 17^{th} dosing cycles each comprises a single dose (C3D1-C17D1) of mosunetuzumab administered on Day 1 of each respective dosing cycle, wherein each of the C3D1-C17D1 is about 30 mg,
wherein the subject has relapsed after, or is refractory to, two or more prior lines of therapy, and
wherein the dosing regimen further comprises:
   (i) administering a corticosteroid, wherein a single dose of the corticosteroid is administered to the subject at least one hour prior to the administration of each of the C1 D1, C1 D2, C1 D3, and C2D1 of mosunetuzumab;
   (ii) administering an anti-histamine, wherein a single dose of the anti-histamine is administered to the subject at least 30 minutes prior to the administration of each of the C1 D1, C1 D2, C1 D3, and C2D1 of mosunetuzumab; and/or
   (iii) administering an anti-pyretic, wherein a single dose of the anti-pyretic is administered to the subject at least 30 minutes prior to the administration of each of the C1 D1, C1 D2, C1 D3, and C2D1 of mosunetuzumab.
132. Mosunetuzumab for use in treating a subject aged about 65 years or older having an R/R NHL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
133. Use of mosunetuzumab for treating a subject aged about 65 years or older having an R/R NHL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
134. Use of mosunetuzumab in the manufacture of a medicament for use in treating a subject aged about 65 years or older having an R/R NHL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
135. The method, mosunetuzumab for use, or use of any one of embodiments 111-134, wherein the two or more prior lines of therapy comprise an anti-CD20 monoclonal antibody, an alkylating agent, or both an anti-CD20 monoclonal antibody and an alkylating agent.
136. The method of any one of embodiments 111-135, wherein the R/R NHL is R/R FL, R/R trFL, or R/R DLBCL.
137. The method, mosunetuzumab for use, or use of embodiment 136, wherein the R/R NHL is R/R FL.
138. The method, mosunetuzumab for use, or use of embodiment 137, wherein the R/R FL is histologically documented as Grade 1, 2, or 3a, but not 3b according to the World Health Organization classification of lymphoid neoplasms (as referenced in Swerdlow S H, et al. Blood 2016; 127:2375-90).
139. The method, mosunetuzumab for use, or use of any one of embodiments 111-138, wherein mosunetuzumab is administered or is to be administered by intravenous infusion.
140. The method, mosunetuzumab for use, or use of any one of embodiments 123-139, wherein:
   (i) a single dose of the corticosteroid is administered or is to be administered prior to the administration of any dose of mosunetuzumab in any of the one or more of any of the dosing cycles after the second dosing cycle;
   (ii) a single dose of the anti-histamine is administered or is to be administered prior to the administration of any dose of mosunetuzumab in any of the one or more of any of the dosing cycles after the second dosing cycle; and/or
   (iii) a single dose of the anti-pyretic is administered or is to be administered prior to the administration of any dose of mosunetuzumab in any of the one or more of any of the dosing cycles after the second dosing cycle.
141. The method, mosunetuzumab for use, or use of any one of embodiments 123-140, wherein:
   (i) the corticosteroid is administered or is to be administered intravenously;
   (ii) the anti-histamine is administered or is to be administered orally or intravenously; and/or
   (iii) the anti-pyretic is administered or is to be administered orally.
142. The method, mosunetuzumab for use, or use of any one of embodiments 123-141, wherein:
   (i) the corticosteroid comprises dexamethasone and is administered or is to be administered at a dose of about 20 mg or the corticosteroid comprises methylprednisolone and is administered or is to be administered at a dose of about 80 mg;
   (ii) the anti-histamine comprises diphenhydramine hydrochloride and is administered or is to be administered at a dose of about 50-100 mg; and/or
   (iii) the anti-pyretic comprises acetaminophen and is administered or is to be administered at a dose of about 500-1000 mg.
143. A method of treating a subject aged about 65 years or older having an R/R FL comprising intravenously administering to the subject mosunetuzumab in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third dosing cycle comprises a single dose (C3D1) of mosunetuzumab administered on Day 1 of the third dosing cycle, wherein the C3D1 is about 30 mg, wherein the subject has relapsed after, or is refractory to, two or more prior lines of therapy.
144. Mosunetuzumab for use in treating a subject aged about 65 years or older having an R/R FL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg, wherein the subject has relapsed after, or is refractory to, two or more prior lines of therapy.
145. Use of mosunetuzumab for treating a subject aged about 65 years or older having an R/R FL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg, wherein the subject has relapsed after, or is refractory to, two or more prior lines of therapy.
146. Use of mosunetuzumab in the manufacture of a medicament for use in treating a subject aged about 65 years or older having an R/R FL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg, wherein the subject has relapsed after, or is refractory to, two or more prior lines of therapy.
147. A method of treating a subject aged about 65 years or older having an R/R FL comprising intravenously administering to the subject mosunetuzumab in a dosing regimen comprising eight 21-day dosing cycles, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third to eighth dosing cycles each comprises a single dose (C3D1-C8D1) of mosunetuzumab administered on Day 1 of each respective dosing cycle, wherein each of the C3D1-C8D1 is about 30 mg, wherein the subject has relapsed after, or is refractory to, two or more prior lines of therapy.
148. Mosunetuzumab for use in treating a subject aged about 65 years or older having an R/R FL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg, wherein the subject has relapsed after, or is refractory to, two or more prior lines of therapy.
149. Use of mosunetuzumab for treating a subject aged about 65 years or older having an R/R FL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg, wherein the subject has relapsed after, or is refractory to, two or more prior lines of therapy.
150. Use of mosunetuzumab in the manufacture of a medicament for use in treating a subject aged about 65 years or older having an R/R FL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg, wherein the subject has relapsed after, or is refractory to, two or more prior lines of therapy.
151. A method of treating a subject aged about 65 years or older having an R/R FL comprising intravenously administering to the subject mosunetuzumab in a dosing regimen comprising 17 21-day dosing cycles, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third to 17^{th} dosing cycles each comprises a single dose (C3D1-C17D1) of mosunetuzumab administered on Day 1 of each respective dosing cycle, wherein each of the C3D1-C17D1 is about 30 mg, wherein the subject has relapsed after, or is refractory to, two or more prior lines of therapy.
152. Mosunetuzumab for use in treating a subject aged about 65 years or older having an R/R FL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg,
wherein the subject has relapsed after, or is refractory to, two or more prior lines of therapy.
153. Use of mosunetuzumab for treating a subject aged about 65 years or older having an R/R FL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg,
wherein the subject has relapsed after, or is refractory to, two or more prior lines of therapy.
154. Use of mosunetuzumab in the manufacture of a medicament for use in treating a subject aged about 65 years or older having an R/R FL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg,
wherein the subject has relapsed after, or is refractory to, two or more prior lines of therapy.
155. A method of treating a subject aged about 65 years or older having an R/R FL comprising intravenously administering to the subject mosunetuzumab in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third dosing cycle comprises a single dose (C3D1) of mosunetuzumab administered on Day 1 of the third dosing cycle, wherein the C3D1 is about 30 mg,
wherein the subject has relapsed after, or is refractory to, two or more prior lines of therapy, and
wherein the dosing regimen further comprises:
   (i) administering a corticosteroid, wherein a single dose of the corticosteroid is administered to the subject at least one hour prior to the administration of each of the C1 D1, C1 D2, C1 D3, and C2D1 of mosunetuzumab;
   (ii) administering an anti-histamine, wherein a single dose of the anti-histamine is administered to the subject at least 30 minutes prior to the administration of each of the C1 D1, C1 D2, C1 D3, and C2D1 of mosunetuzumab; and/or
   (iii) administering an anti-pyretic, wherein a single dose of the anti-pyretic is administered to the subject at least 30 minutes prior to the administration of each of the C1 D1, C1 D2, C1 D3, and C2D1 of mosunetuzumab.
156. Mosunetuzumab for use in treating a subject aged about 65 years or older having an R/R FL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
157. Use of mosunetuzumab for treating a subject aged about 65 years or older having an R/R FL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
158. Use of mosunetuzumab in the manufacture of a medicament for use in treating a subject aged about 65 years or older having an R/R FL, wherein mosunetuzumab is to be administered intravenously to the subject in a dosing regimen comprising at least a first 21-day dosing cycle, a second 21-day dosing cycle, and a third 21-day dosing cycle, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third dosing cycle comprises a single dose (C3D1) of mosunetuzumab to be administered on Day 1 of the third dosing cycle, wherein the C3D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
159. A method of treating a subject aged about 65 years or older having an R/R FL comprising intravenously administering to the subject mosunetuzumab in a dosing regimen comprising eight 21-day dosing cycles, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third to eighth dosing cycles each comprises a single dose (C3D1-C8D1) of mosunetuzumab administered on Day 1 of each respective dosing cycle, wherein each of the C3D1-C8D1 is about 30 mg,
wherein the subject has relapsed after, or is refractory to, two or more prior lines of therapy, and
wherein the dosing regimen further comprises:
   (i) administering a corticosteroid, wherein a single dose of the corticosteroid is administered to the subject at least one hour prior to the administration of each of the C1 D1, C1 D2, C1 D3, and C2D1 of mosunetuzumab;
   (ii) administering an anti-histamine, wherein a single dose of the anti-histamine is administered to the subject at least 30 minutes prior to the administration of each of the C1 D1, C1 D2, C1 D3, and C2D1 of mosunetuzumab; and/or
   (iii) administering an anti-pyretic, wherein a single dose of the anti-pyretic is administered to the subject at least 30 minutes prior to the administration of each of the C1 D1, C1 D2, C1 D3, and C2D1 of mosunetuzumab.
160. Mosunetuzumab for use in treating a subject aged about 65 years or older having an R/R FL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
161. Use of mosunetuzumab for treating a subject aged about 65 years or older having an R/R FL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
162. Use of mosunetuzumab in the manufacture of a medicament for use in treating a subject aged about 65 years or older having an R/R FL, wherein mosunetuzumab is to be administered intravenously to the subject in a dosing regimen comprising eight 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to eighth dosing cycles each comprises a single dose (C3D1-C8D1) of mosunetuzumab to be administered on Day 1 of each respective dosing cycle, wherein each of the C3D1-C8D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
163. A method of treating a subject aged about 65 years or older having an R/R FL comprising intravenously administering to the subject mosunetuzumab in a dosing regimen comprising 17 21-day dosing cycles, wherein:
   (a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab administered on Day 1 of the second dosing cycle, wherein the C2D1 is about 60 mg; and
   (c) the third to 17^{th} dosing cycles each comprises a single dose (C3D1-C17D1) of mosunetuzumab administered on Day 1 of each respective dosing cycle, wherein each of the C3D1-C17D1 is about 30 mg,
wherein the subject has relapsed after, or is refractory to, two or more prior lines of therapy, and
wherein the dosing regimen further comprises:
   (i) administering a corticosteroid, wherein a single dose of the corticosteroid is administered to the subject at least one hour prior to the administration of each of the C1 D1, C1 D2, C1 D3, and C2D1 of mosunetuzumab;
   (ii) administering an anti-histamine, wherein a single dose of the anti-histamine is administered to the subject at least 30 minutes prior to the administration of each of the C1 D1, C1 D2, C1 D3, and C2D1 of mosunetuzumab; and/or
   (iii) administering an anti-pyretic, wherein a single dose of the anti-pyretic is administered to the subject at least 30 minutes prior to the administration of each of the C1 D1, C1 D2, C1 D3, and C2D1 of mosunetuzumab.
164. Mosunetuzumab for use in treating a subject aged about 65 years or older having an R/R FL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
165. Use of mosunetuzumab for treating a subject aged about 65 years or older having an R/R FL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
166. Use of mosunetuzumab in the manufacture of a medicament for use in treating a subject aged about 65 years or older having an R/R FL, wherein mosunetuzumab is to be administered intravenously to the subject in a dosing regimen comprising 17 21-day dosing cycles, wherein:
   (a) the first three-dose dosing cycle comprises a first dose (C1D1), a second dose (C1D2), and a third dose (C1D3) of mosunetuzumab to be administered on Days 1, 8, and 15, respectively, of the first dosing cycle, wherein the C1 D1 is about 1 mg, the C1 D2 is about 2 mg, and the C1 D3 is about 60 mg;
   (b) the second dosing cycle comprises a single dose (C2D1) of mosunetuzumab to be administered on Day 1 of the second dosing cycle, wherein the C2D1 is about 60 mg; and
   (c) the third to 17^{th} dosing cycles each comprises a single dose (C3D1-C17D1) of mosunetuzumab to be administered on Day 1 of each respective dosing cycle, wherein each of the C3D1-C17D1 is about 30 mg,
wherein the subject has relapsed after, or is refractory to, two or more prior lines of therapy, and
wherein the dosing regimen further comprises:
   (i) administration of a corticosteroid, wherein a single dose of the corticosteroid is to be administered to the subject at least one hour prior to the administration of each of the C1 D1, C1 D2, C1 D3, and C2D1 of mosunetuzumab;
   (ii) administration of an anti-histamine, wherein a single dose of the anti-histamine is to be administered to the subject at least 30 minutes prior to the administration of each of the C1 D1, C1 D2, C1 D3, and C2D1 of mosunetuzumab; and/or
   (iii) administration of an anti-pyretic, wherein a single dose of the anti-pyretic is to be administered to the subject at least 30 minutes prior to the administration of each of the C1 D1, C1 D2, C1 D3, and C2D1 of mosunetuzumab.
167. The method, mosunetuzumab for use, or use of any one of embodiments 143-155, wherein the two or more prior lines of therapy comprise an anti-CD20 monoclonal antibody, an alkylating agent, or both an anti-CD20 monoclonal antibody and an alkylating agent.
168. The method, mosunetuzumab for use, or use of embodiment 167, wherein the R/R FL is histologically documented as Grade 1, 2, or 3a, but not 3b according to the World Health Organization classification of lymphoid neoplasms (as referenced in Swerdlow S H, et al. Blood 2016; 127:2375-90).
169. The method, mosunetuzumab for use, or use of any one of embodiments 143-168, wherein mosunetuzumab is administered or is to be administered by intravenous infusion.
170. The method, mosunetuzumab for use, or use of any one of embodiments 155-169, wherein:
   (i) a single dose of the corticosteroid is administered or is to be administered prior to the administration of any dose of mosunetuzumab in any of the one or more of any of the dosing cycles after the second dosing cycle;
   (ii) a single dose of the anti-histamine is administered or is to be administered prior to the administration of any dose of mosunetuzumab in any of the one or more of any of the dosing cycles after the second dosing cycle; and/or
   (iii) a single dose of the anti-pyretic is administered or is to be administered prior to the administration of any dose of mosunetuzumab in any of the one or more of any of the dosing cycles after the second dosing cycle.
171. The method, mosunetuzumab for use, or use of any one of embodiments 155-170, wherein:
   (i) the corticosteroid is administered or is to be administered intravenously;
   (ii) the anti-histamine is administered or is to be administered orally or intravenously; and/or
   (iii) the anti-pyretic is administered or is to be administered orally.
172. The method, mosunetuzumab for use, or use of any one of embodiments 155-171, wherein:
   (i) the corticosteroid comprises dexamethasone and is administered or is to be administered at a dose of about 20 mg or the corticosteroid comprises methylprednisolone and is administered or is to be administered at a dose of about 80 mg;
   (ii) the anti-histamine comprises diphenhydramine hydrochloride and is administered or is to be administered at a dose of about 50-100 mg; and/or
   (iii) the anti-pyretic comprises acetaminophen and is administered or is to be administered at a dose of about 500-1000 mg.

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating a subject aged about 65 years or older having a relapsed or refractory (R/R) non-Hodgkin's lymphoma (NHL) comprising intravenously administering to the subject mosunetuzumab in a dosing regimen comprising at least a first dosing cycle, a second dosing cycle, and a third dosing cycle, wherein:
(a) the first three-dose dosing cycle comprises a first dose (C1D1), a second dose (C1D2), and a third dose (C1D3) of mosunetuzumab, wherein the C1 D1 is about 1 mg, the C1 D2 is about 2 mg, and the C1 D3 is about 60 mg;
(b) the second dosing cycle comprises a single dose (C2D1) of mosunetuzumab, wherein the C2D1 is about 60 mg; and
(c) the third dosing cycle comprises a single dose (C3D1) of mosunetuzumab, wherein the C3D1 is about 30 mg.

2. The method of claim 1, wherein:
(a) the subject has relapsed after, or is refractory to, two or more prior lines of therapy;
(b) the R/R NHL is R/R follicular lymphoma (FL), R/R transformed FL (trFL), or R/R diffuse large B cell lymphoma (DLBCL);
(c) the first, second, and third dosing cycles are 21-day dosing cycles;
(d) the method comprises administering:
(i) the C1 D1, the C1 D2, and the C1 D3 on Days 1, 8, and 15, respectively, of the first dosing cycle;
(ii) the C2D1 on Day 1 of the second dosing cycle; and/or
(iii) the C3D1 on Day 1 of the third dosing cycle;
(e) the dosing regimen further comprises one or more additional dosing cycles; and/or
(f) mosunetuzumab is administered by intravenous infusion.

3. The method of claim 2, wherein:
(a) the two or more prior lines of therapy comprise an anti-CD20 monoclonal antibody, an alkylating agent, or both an anti-CD20 monoclonal antibody and an alkylating agent,
(b) the R/R NHL is R/R FL; and/or
(c) the dosing regimen comprises five to 14 additional dosing cycles.

4-5. (canceled)

6. The method of claim 3, wherein:
(a) the R/R FL is histologically documented as Grade 1, 2, or 3a, but not 3b according to the World Health Organization classification of lymphoid neoplasms (as referenced in Swerdlow S H, et al. Blood 2016; 127:2375-90); and/or
(b) the dosing regimen comprises five or 14 additional dosing cycles.

7-14. (canceled)

15. The method of claim 2, wherein:
(a) the one or more additional dosing cycles are each 21-day dosing cycles; and/or
(b) each of the one or more of the additional dosing cycles comprises an additional single dose of mosunetuzumab.

16. (canceled)

17. The method of claim 15, wherein the additional single dose of mosunetuzumab is administered to the subject on Day 1 of each respective additional dosing cycle and/or the additional single dose of mosunetuzumab is about 30 mg.

18-20. (canceled)

21. A method of treating a population of subjects aged about 65 years or older having an R/R NHL comprising intravenously administering to each subject mosunetuzumab in a dosing regimen comprising eight 21-day dosing cycles, wherein:
(a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
(b) the second dosing cycle comprises a single dose (C2D1) of mosunetuzumab administered on Day 1 of the second dosing cycle, wherein the C2D1 is about 60 mg; and
(c) the third to eighth dosing cycles each comprises a single dose (C3D1-C8D1) of mosunetuzumab administered on Day 1 of each respective dosing cycle, wherein each of the C3D1-C8D1 is about 30 mg.

22-28. (canceled)

29. The method of claim 21, wherein:
(a) the objective response rate in the population of subjects is ≥65%;
(b) the complete response rate in the population of subjects is ≥50%;
(c) ≥40% of subjects having an objective response maintained remission for 18 months;
(d) ≥50% of subjects having a complete response maintained complete remission for 18 months;
(e) the median duration of response in the population of subjects is ≥9 months;
(f) the median duration of complete response in the population of subjects is ≥12 months;
(g) ≥35% of subjects have a progression-free survival of 18 months;
(h) the median progression-free survival in the population of subjects is ≥12 months;
(i) the rate of serious adverse events, excluding Grade 5 malignant neoplasm progression, in the population of subjects is ≤45%;
(j) the rate of serious adverse events related to mosunetuzumab in the population of subjects is ≤40%;
(k) the rate of cytokine release syndrome having a grade of 3 or higher (as defined by the American Society for Transplantation and Cellular Therapy, 2018; ASTCT) in the population of subjects is ≤5%; and/or
(l) the rate of cytokine release syndrome of any grade (as defined by the ASTCT) in the population of subjects is ≤40%.

30. The method of claim 29, wherein:
(a) the objective response rate in the population of subjects is ≥75%;
(b) the complete response rate in the population of subjects is ≥60%;
(c) ≥50% of subjects having an objective response maintained remission for 18 months;
(d) ≥60% of subjects having a complete response maintained complete remission for 18 months;
(e) the median duration of response in the population of subjects is ≥15 months;
(f) the median duration of complete response in the population of subjects is ≥16 months;
(g) ≥45% of subjects have a progression-free survival of 18 months;
(h) the median progression-free survival in the population of subjects is ≥17 months;
(i) the rate of serious adverse events, excluding Grade 5 malignant neoplasm progression, in the population of subjects is ≤40%;
(j) the rate of serious adverse events related to mosunetuzumab in the population of subjects is ≤30%;
(k) the rate of cytokine release syndrome having a grade of 3 or higher (as defined by the ASTCT) in the population of subjects is ≤3%; and/or
(l) the rate of cytokine release syndrome of any grade (as defined by the ASTCT) in the population of subjects is ≤30%.

31. The method of claim 30, wherein the objective response rate in the population of subjects is ≥85% and/or the complete response rate in the population of subjects is ≥70%.

32-55. (canceled)

56. A method of achieving an objective response, a complete response, or progression-free survival in a subject aged about 65 years or older having an R/R NHL, wherein the method comprises intravenously administering to the subject mosunetuzumab in a dosing regimen comprising eight 21-day dosing cycles, wherein:
(a) the first three-dose dosing cycle comprises a first dose (C1D1), a second dose (C1D2), and a third dose (C1D3) of mosunetuzumab administered on Days 1, 8, and 15, respectively, of the first dosing cycle, wherein the C1 D1 is about 1 mg, the C1 D2 is about 2 mg, and the C1 D3 is about 60 mg;
(b) the second dosing cycle comprises a single dose (C2D1) of mosunetuzumab administered on Day 1 of the second dosing cycle, wherein the C2D1 is about 60 mg; and
(c) the third to eighth dosing cycles each comprises a single dose (C3D1-C8D1) of mosunetuzumab administered on Day 1 of each respective dosing cycle, wherein each of the C3D1-C8D1 is about 30 mg.

57-63. (canceled)

64. The method of claim 56, wherein:
(a) the subject achieves an objective response;
(b) the subject achieves a complete response; and/or
(c) the progression-free survival is maintained for ≥12 months.

65-66. (canceled)

67. The method of claim 64, wherein the progression-free survival is maintained for ≥18 months.

68. The method of claim 1, wherein the method further comprises administering to the subject or to each subject in the population of subjects one or more additional therapeutic agents.

69. The method of claim 68, wherein the one or more additional therapeutic agents comprises;
(a) tocilizumab,
(b) a corticosteroid;
(c) an anti-histamine; and/or
(d) an anti-pyretic.

70. (canceled)

71. The method of claim 69, wherein:
(a) the corticosteroid:
(i) comprises dexamethasone administered at a dose of about 20 mg or methylprednisolone administered at a dose of about 80 mg;
(ii) is administered to the subject or to each subject in the population of subjects at least one hour prior to the administration of any dose of mosunetuzumab;
(iii) is only administered during dosing cycle 1 or dosing cycle 2; and/or
(iv) is administered intravenously;
(b) the anti-histamine:
(i) is administered to the subject or to each subject in the population of subjects at least 30 minutes prior to the administration of any dose of mosunetuzumab;
(ii) is only administered during dosing cycle 1 or dosing cycle 2;
(iii) is administered orally or intravenously; and/or
(iv) comprises diphenhydramine hydrochloride and is administered at a dose of about 50-100 mg; and/or
(c) the anti-pyretic:
(i) is administered to the subject or to each subject in the population of subjects at least 30 minutes prior to the administration of any dose of mosunetuzumab;
(ii) is only administered during dosing cycle 1 or dosing cycle 2;
(iii) is administered orally; and/or
(iv) comprises acetaminophen and is administered at a dose of about 500-1000 mg.

72-100. (canceled)
